(19) United States
(12) Patent Application Publication
(10) Pub. No.: US 2024/0150508 A1
(43) Pub. Date: May 9, 2024

(54) PHOTO-SWITCHABLE CHEMISTRY FOR REVERSIBLE HYDROGELS AND REUSABLE FLOW CELLS

(71) Applicants: Illumina, Inc., San Diego, CA (US); Illumina Cambridge Limited, Cambridge (GB); Illumina Singapore Pte. Ltd., Singapore (SG)

(72) Inventors: Nam Nguyen, Cambridge (GB); Xavier von Hatten, Cambridge (GB); Wayne George, Ilford (GB); Gianluca Artioli, Cambridge (GB); Brian Mather, San Diego, CA (US); Johan Basuki, Singapore (SG); Shima Gholizadeh, Los Angeles, CA (US)

(73) Assignees: Illumina, Inc., San Diego, CA (US); Illumina Cambridge Limited, Cambridge (GB); Illumina Singapore Pte. Ltd., Singapore (SG)

(21) Appl. No.: 18/482,417

(22) Filed: Oct. 6, 2023

Related U.S. Application Data
(60) Provisional application No. 63/378,943, filed on Oct. 10, 2022.

(51) Int. Cl. C08F 220/56; C08J 3/24; C08J 3/28

(52) U.S. Cl. CPC C08F 220/56; C08J 3/245; C08J 3/28; C08F 2810/20; C08F 2810/40; C08J 2333/26

(57) ABSTRACT

In some examples, novel photochemically-reversible hydrogels and nanogel particles are described comprising copolymer chains including at least one reactive alkene or reactive 1,4-diene end group capable of [2+2] or [2+2+2+2] photodimerization, respectively, at wavelengths >270 nm. In various examples, the photochemically-reversible hydrogels comprise copolymer chains including at least one —N₃, —C≡CH or —CO₂H end group for dual functionality and/or pH responsiveness. For nucleic acid sequencing, amplification primers are grafted to photochemically-reversible hydrogels or nanogel particles reversibly bound to surfaces within a flow cell. After sequencing is complete, the photochemically-reversible hydrogel or nanogel particles is/are removable from the flow cell surfaces by irradiation, enabling the flow cell to be reusable.

### CROSS-REFERENCE TO RELATED APPLICATION

This application claims the benefit of U.S. Provisional Patent Application No. 63/378,943, filed Oct. 10, 2022 and entitled “Photo-switchable Chemistry for Reversible Hydrogels and Reusable Flow Cells,” the disclosure of which is hereby incorporated by reference in its entirety.

### FIELD

The present disclosure generally relates to nucleic acid sequencing methods and devices and in particular to photochemically-reversible hydrogels and nanogel particles usable in sequencing by synthesis (SBS) methods.

### BACKGROUND

Nucleic acid sequencing continues to be an important tool in a multitude of diverse fields, from ancestry to law enforcement to medical diagnostics. Sequencing methods today are rapid and cost effective. Nonetheless, there is a continuing need to drive genetic sequencing costs down even further, such as by streamlining individual process steps in sequencing methods and improving various devices, such as flow cells used in Sequencing by Synthesis (SBS).

### SUMMARY

Photochemically-reversible hydrogels and photochemically-reversible nanogel particles having photo-switchable chemistry for nucleic acid sequencing systems are provided herein.

For example, as provided herein, certain polymeric hydrogels and nanogel particles having photochemical reversibility may be used to replace hydrogel coatings in flow cells for Sequencing by Synthesis (SBS). Polymeric hydrogels and nanogel particles having photochemical reversibility may be used to improve many aspects of an SBS method, such as enabling sequencing flow cells to be reusable after hydrogel or nanogel particle removal.

In various examples provided herein, certain polymeric hydrogels and nanogel particles having photochemical reversibility may be attached to flow cell surfaces upon exposure to light of frequency hυ1>270 nm and may be detached from flow cell surfaces upon exposure to light of frequency hυ2<300 nm.

In various examples provided herein, certain polymeric hydrogels and nanogel particles having photochemical reversibility comprise copolymer chains that include at least one reactive alkene or reactive 1,4-diene end group capable of [2+2] or [2+2+2+2] photodimerization, respectively, at wavelengths hυ1>270 nm.

In various examples provided herein, a nanogel particle having photochemical reversibility functions as a replacement for a nano-well in a flow cell, thus eliminating the need to configure nano-wells in the flow cells for SBS. Other examples may include an operation trapping nanogel particles in nano-wells configured in a flow cell.

In various examples provided herein, sequencing on a nanogel particle versus a hydrogel surface improves the monoclonality of the clustering of multiple copies of a sequencing template. For example, confining the clustering to a nano scale particle may improve signal to noise ratio, error rate, and overall quality and coverage of genome during sequencing.

In various examples, nanogel particles having photochemical reversibility disclosed herein also exhibit dual functionality through the presence of at least two types of reactive end groups on copolymer chains within the nanogel particles. In various examples, nanogel particles having photochemical reversibility and dual functionality comprise copolymer chains that include at least one reactive alkene or reactive 1,4-diene end group capable of [2+2] or [2+2+2+2] photodimerization, respectively, at wavelengths hυ1>270 nm, and copolymer chains having at least one of azide end groups and carboxylic acid end groups.

In various examples, nanogel particles having photochemical reversibility disclosed herein also exhibit dual responsiveness, namely temperature and pH responsiveness. Temperature responsiveness is due in part to copolymer chains having sections of poly(N-isopropylacrylamide) units, and pH responsiveness is due in part to copolymer chains having carboxylic acid end groups.

Dual functionality and dual responsiveness (temperature/pH) characteristics of nanogel particles, such as provided herein, allows initial attachment of alkyne-functionalized amplification primers onto the particles using, for example, —N₃ functionality on the particles, while pH responsiveness enhances chemical capture onto flow cell surfaces using bioconjugation techniques. These amplification primer-functionalized nanogel particles are demonstrated to support on-board particle clustering and SBS sequencing.

In various examples, a hydrogel polymer comprises copolymer chains further comprising:
a first recurring unit of Formula (I):

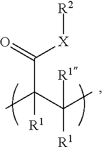

wherein
each of R¹, R^{1′}, and R^{1″} is independently selected from H, halogen, alkyl, alkoxy, alkenyl, alkynyl, cycloalkyl, aryl, heteroaryl, or heterocyclyl;
X is —O— or —NH—; and
R² is —CH₂—C≡CH, or R² has a structure:

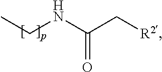

wherein R^{2′} is —NH₂, alkyl, alkoxy, alkenyl, alkynyl, or optionally substituted variants thereof, or halogen, —N₃, —OH, —C(O)H, —NH═NH₂, —SCN, —CO₂H, —SH, glycidyl, epoxy, aziridine, triazoline, or —C≡CH; and
p is an integer of 1 to 50; and
a second recurring unit of Formula (II),

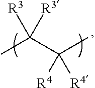

wherein:
each of R³, R^{3′}, R⁴, and R^{4′} is independently selected from —H, —R⁵, —OR⁵, —CO₂R⁵, —C(O)R⁵, —OC(O)R⁵, —C(O)NR⁶R⁷, —NR⁶R⁷ or a substructure of Formula (III),

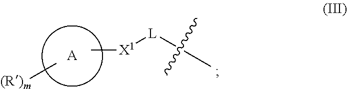

R⁵ is —H, —OH, alkyl, cycloalkyl, hydroxyalkyl, aryl, heteroaryl, or heterocyclyl;
each of R⁶ and R⁷ is independently selected from —H and alkyl;
A is an aryl or a thymidinyl moiety;
R′ is —H, alkyl, alkoxy, alkenyl, alkynyl, aryl, heterocyclyl or optionally substituted variants thereof, or halogen, —N₃, —OH, —C(O)H, —NH═NH₂, —SCN, —CO₂H, —SH, glycidyl, epoxy, or two R′ groups, when bonded to adjacent atoms on ring A and taken together with ring A, form a coumarinyl, an anthracenyl, an acenaphthylenyl, a thianaphthenyl-1-oxide or a thianaphthenyl -1,1-dioxide moiety;
X¹ is a bond, —(CH₂)_{q}—, —O— or —NH—;
L is a bivalent linker having a structure —(CH₂)_{q}—X²—C(═O)— or —(CH₂CH₂O)_{q}—X²—C(═O)—;
X² is —O— or —NH—;
m is an integer from 1 to 9; and
q is an integer from 0 to 50;
wherein at least some of the copolymer chains include at least one reactive alkene or reactive 1,4-diene end group capable of [2+2] or [2+2+2+2] photodimerization, respectively, at wavelengths >270 nm.

In various examples, at least some of the copolymer chains include at least one N₃, —C≡CH or —CO₂H end group.

In various examples, Formula (III) comprises the subgenus structure of Formula (IV):

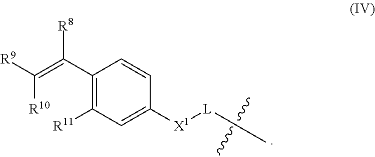

wherein each of R⁸ and R⁹ is —H, alkyl, cycloalkyl, hydroxyalkyl, aryl, heteroaryl, or heterocyclyl; each R¹⁰ and R¹¹ is independently —H, alkyl, cycloalkyl, hydroxyalkyl, aryl, heteroaryl, or heterocyclyl, or R¹⁰ is —C(═O)—, R¹¹ is —O—, and R¹⁰ and R¹¹ are bonded together such that Formula (IV) comprises a substituted coumarin moiety.

In various examples, the recurring unit of Formula (I) is:

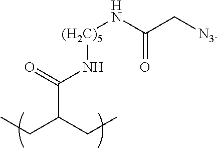

In various examples, the recurring unit of Formula (II) is:

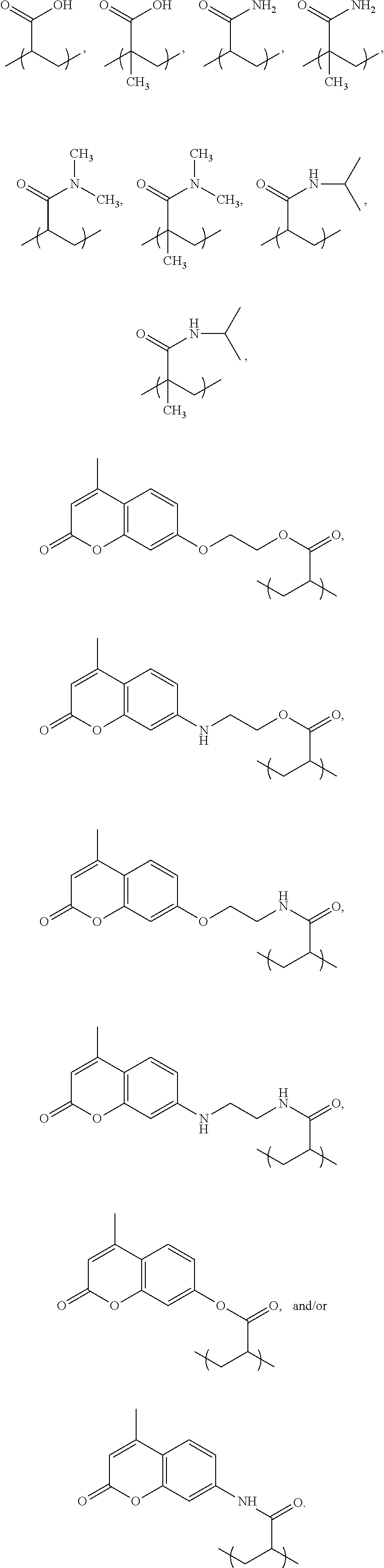

In various examples, the recurring unit of Formula (II) is:

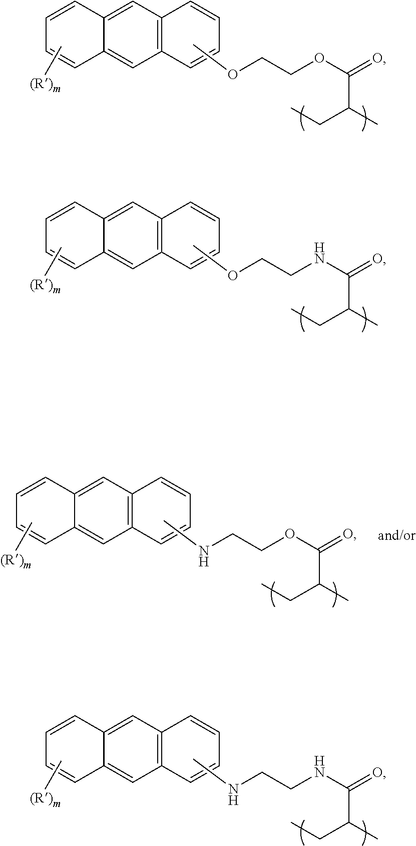

wherein R′ is —H, alkyl, alkoxy, alkenyl, alkynyl, aryl, heterocyclyl or optionally substituted variants thereof, or halogen, —N₃, —OH, —C(O)H, —NH═NH₂, —SCN, —CO₂H, —SH, glycidyl, epoxy; and,
m is an integer from 1 to 9.

In various examples, the recurring unit of Formula (II) is:

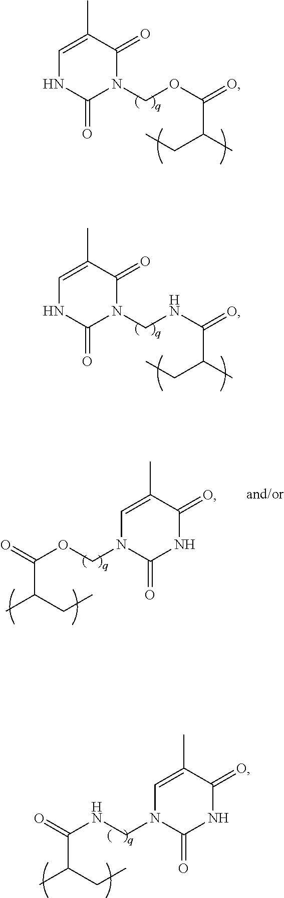

wherein q is an integer from 0 to 50.

In various examples, the recurring unit of Formula (II) is

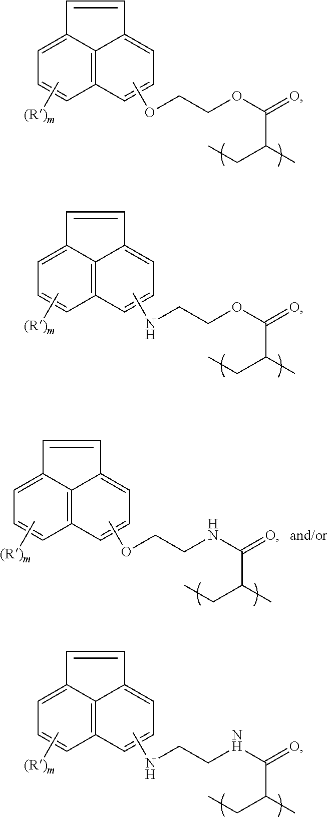

wherein R′ is —H, alkyl, alkoxy, alkenyl, alkynyl, aryl, heterocyclyl or optionally substituted variants thereof, or halogen, —N₃, —OH, —C(O)H, —NH═NH₂, —SCN, —CO₂H, —SH, glycidyl, epoxy; and,
m is an integer from 1 to 7.

In various examples, the recurring unit of Formula (II) is:

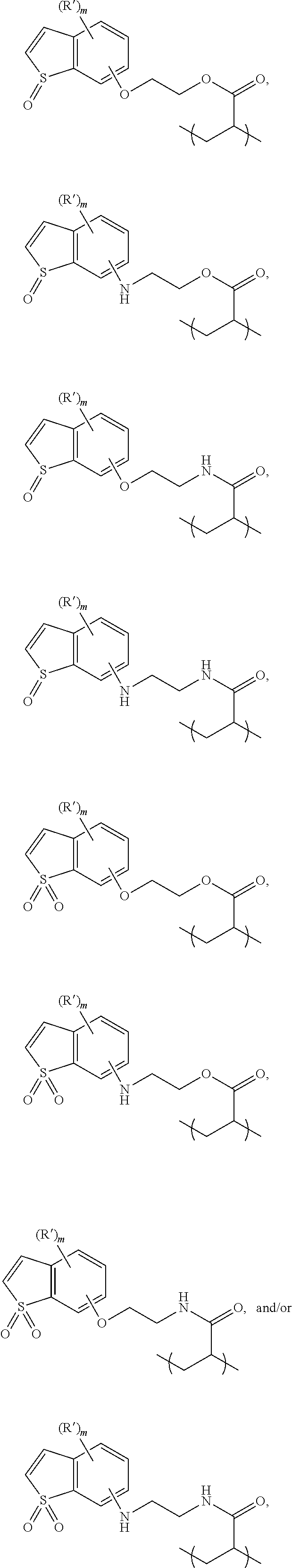

wherein R′ is —H, alkyl, alkoxy, alkenyl, alkynyl, aryl, heterocyclyl or optionally substituted variants thereof, or halogen, —N₃, —OH, —C(O)H, —NH═NH₂, —SCN, —CO₂H, —SH, glycidyl, epoxy; and,
m is an integer from 1 to 5.

In various examples, the recurring unit of Formula (II) is:

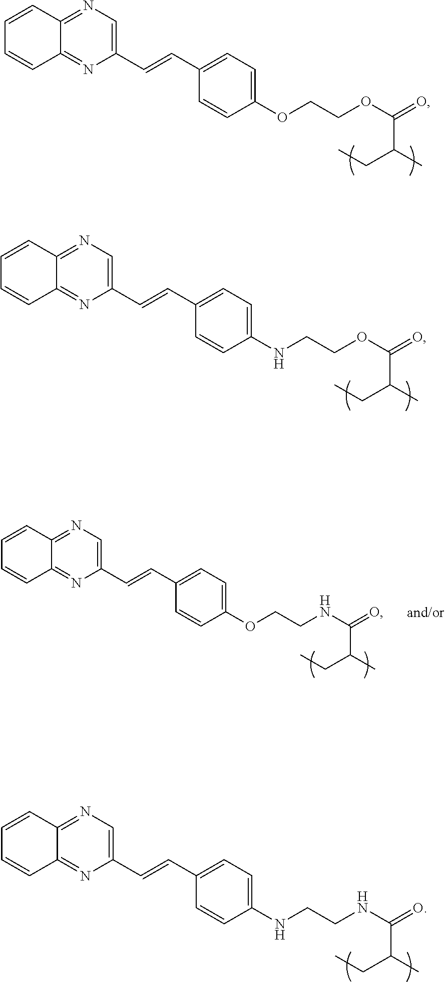

In various examples, either R⁹ or R¹⁰ is —CO₂H, such that Formula (IV) is a cis- or trans-cinnamyl acid moiety.

In various examples, R⁹ or R¹⁰ is aryl, such that Formula (IV) is a cis- or trans-stilbene moiety.

In various examples, a hydrogel polymer is derived from a monomer mixture comprising: 7-((2-methacryloyloxy)ethoxy)-4-methylcoumarin; N-isopropylacrylamide; N-(5-(2-azidoacetamido)pentyl)acrylamide; and acrylic acid.

In various examples, a hydrogel polymer is derived from a monomer mixture comprising: 7-((2-methacryloyloxy)ethoxy)-4-methylcoumarin; N,N-dimethylacrylamide; N-(5-(2-azidoacetamido)pentyl)acrylamide; and acrylic acid.

In various examples, a hydrogel polymer is derived from a monomer mixture comprising: 7-((2-acrylamido)ethoxy)-4-methylcoumarin; N-isopropylacrylamide; N-(5-(2-azidoacetamido)pentyl)acrylamide; and acrylic acid.

In various examples, a hydrogel polymer is derived from a monomer mixture comprising: 7-((2-acrylamido)ethoxy)-4-methylcoumarin; N,N-dimethylacrylamide; N-(5-(2-azidoacetamido)pentyl)acrylamide; and acrylic acid.

In various examples, a hydrogel polymer is derived derived from a monomer mixture comprising: 7-(acrylamido)-4-methylcoumarin; N-isopropylacrylamide; N-(5-(2-azidoacetamido)pentyl)acrylamide; and acrylic acid.

In various examples, a hydrogel polymer is derived from a monomer mixture comprising: 7-(acrylamido)-4-methylcoumarin; N,N-dimethylacrylamide; N-(5-(2-azidoacetamido)pentyl)acrylamide; and acrylic acid.

In various examples, a hydrogel polymer is derived from a monomer mixture comprising: 7-(methacrylamido)-4-methylcoumarin; N-isopropylacrylamide; N-(5-(2-azidoacetamido)pentyl)acrylamide; and acrylic acid.

In various examples, a hydrogel polymer is derived from a monomer mixture comprising: 7-(methacrylamido)-4-methylcoumarin; N,N-dimethylacrylamide; N-(5-(2-azidoacetamido)pentyl)acrylamide; and acrylic acid.

In various examples, a hydrogel polymer is derived from a monomer mixture comprising: 7-(acryloyloxy)-4-methylcoumarin; N-isopropylacrylamide; N-(5-(2-azidoacetamido)pentyl)acrylamide; and acrylic acid.

In various examples, a hydrogel polymer is derived from a monomer mixture comprising: 7-(acryloyloxy)-4-methylcoumarin; N,N-dimethylacrylamide; N-(5-(2-azidoacetamido)pentyl)acrylamide; and acrylic acid.

In various examples, a hydrogel polymer is derived from a monomer mixture comprising: 7-(methacryloyloxy)-4-methylcoumarin; N-isopropylacrylamide; N-(5-(2-azidoacetamido)pentyl)acrylamide; and acrylic acid.

In various examples, a hydrogel polymer is derived from a monomer mixture comprising: 7-(methacryloyloxy)-4-methylcoumarin; N,N-dimethylacrylamide; N-(5-(2-azidoacetamido)pentyl)acrylamide; and acrylic acid.

In various examples, the monomer mixture further comprises a multi-functional compound selected from the group consisting of N,N′-methylenebisacrylamide, N,N′-methylenebismethacrylamide, polyethylene glycol diacrylate, polyethylene glycol dimethacrylate, N-vinylacrylamide, glycidyl acrylate, divinylbenzene, tetraallyl ammonium chloride, and mixtures thereof.

In various examples, the hydrogel polymer is in the form of nanogel particles.

In various examples, the hydrogel polymer further comprises amplification primers conjugated thereon.

In various examples, each conjugation between an amplification primer and the hydrogel polymer comprises click-chemistry between a terminal alkyne substituent on the amplification primer and an azide group on an end of a respective copolymer chain or click-chemistry between a terminal azide substituent on the amplification primer and an alkyne group on an end of a respective copolymer chain.

In various examples, at least some of the copolymer chains are crosslinked by photo-dimerization between the reactive alkene or reactive 1,4-diene end groups capable of [2+2] or [2+2+2+2] photodimerization, respectively, at wavelengths >270 nm.

In various examples, a substrate having a surface comprises a hydrogel polymer covalently attached thereto, wherein the hydrogel polymer includes a plurality of copolymer chains further comprising a recurring unit of Formula (I) and a recurring unit of Formula (II);

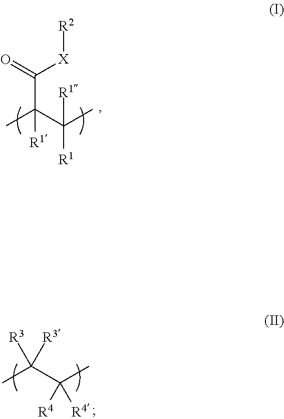

wherein:
each of R¹, R^{1′}, and R^{1″} is independently selected from H, halogen, alkyl, alkoxy, alkenyl, alkynyl, cycloalkyl, aryl, heteroaryl, or heterocyclyl;
X is —O— or —NH—;
R² is —CH₂—C≡CH, or R² has a structure:

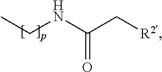

R^{2′} is —NH₂, alkyl, alkoxy, alkenyl, alkynyl, or optionally substituted variants thereof, or halogen, —N₃, —OH, —C(O)H, —NH═NH₂, —SCN, —CO₂H, —SH, glycidyl, epoxy, aziridine, triazoline, or —C≡CH;
p is an integer of 1 to 50;
each of R³, R^{3′}, R⁴, and R^{4′}is independently selected from —H, —R⁵, —OR⁵, —CO₂R⁵, —C(O)R⁵, —OC(O)R⁵, —C(O)NR⁶R⁷, —NR⁶R⁷ or a substructure of Formula (III),

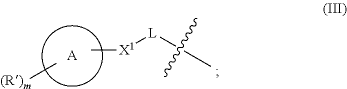

R⁵ is —H, —OH, alkyl, cycloalkyl, hydroxyalkyl, aryl, heteroaryl, or heterocyclyl;
each of R⁶ and R⁷ is independently selected from —H and alkyl;
A is an aryl or a thymidinyl moiety;
R′ is —H, alkyl, alkoxy, alkenyl, alkynyl, aryl, heterocyclyl or optionally substituted variants thereof, or halogen, —N₃, —OH, —C(O)H, —NH═NH₂, —SCN, —CO₂H, —SH, glycidyl, epoxy, or two R′ groups, when bonded to adjacent atoms on ring A and taken together with ring A, form a coumarinyl, an anthracenyl, an acenaphthylenyl, a thianaphthenyl-1-oxide or a thianaphthenyl -1,1-dioxide moiety;
X¹ is a bond, —(CH₂)_{q}—, —O— or —NH—;
L is a bivalent linker having a structure —(CH₂)_{q}—X²—C(═O)— or —(CH₂CH₂O)_{q}—X²—C(═O)—;
X² is —O— or —NH—;
m is an integer from 1 to 9; and
q is an integer from 0 to 50;
wherein at least some of the copolymer chains include at least one —N₃, —C≡CH or —CO₂H end group; and wherein at least some of the copolymer chains include at least one reactive alkene or reactive 1,4-diene end group capable of [2+2] or [2+2+2+2] photodimerization, respectively, at wavelengths >270 nm.

In various examples, the covalent attachment between the substrate and the hydrogel polymer comprise photo-dimerized linkages between the reactive alkene or reactive 1,4-diene end groups of the copolymer chains capable of [2+2] or [2+2+2+2] photodimerization and corresponding reactive alkene or reactive 1,4-diene groups disposed on the substrate surface.

In various examples, the photo-dimerized linkages comprise at least one of coumarin dimers, anthracene dimers, thymidine dimers, cinnamic acid dimers, stilbene dimers, acenaphthylene dimers, 2-methylthianaphthene-1-oxide dimers, 2-methylthianaphthene-1,1-dioxide dimers, or styryl quinoxaline dimers. In various examples, the hydrogel polymer is in the form of nanogel particles. In various examples, the hydrogel polymer further comprises amplification primers conjugated thereon.

In various examples, each conjugation between an amplification primer and the hydrogel polymer comprises click-chemistry between a terminal alkyne substituent on the amplification primer and an azide end group on a respective copolymer chain or click-chemistry between a terminal azide substituent on the amplification primer and an alkyne end group on a respective copolymer chain.

In various examples, at least some of the copolymer chains of the hydrogel polymer are crosslinked by photo-dimerization between the reactive alkene or reactive 1,4-diene end groups capable of [2+2] or [2+2+2+2] photodimerization, respectively, at wavelengths >270 nm.

In various examples, a flow cell comprises the substrate described herein above.

In various examples, a method of synthesizing a hydrogel polymer having crosslinked copolymer chains comprises:
(1) reacting an aqueous dispersion of a monomer mixture comprising (a) 7-((2-acryloyloxy)ethoxy)-4-methylcoumarin, 7-((2-methacryloyloxy)ethoxy)-4-methylcoumarin, 7-((2-acrylamido)ethoxy)-4-methylcoumarin, 7-((2-methacrylamido)ethoxy)-4-methylcoumarin, 7-((2-acryloyloxy)aminoethyl)-4-methylcoumarin, 7-((2-methacryloyloxy) aminoethyl)-4-methylcoumarin, 7-((2-acrylamido) aminoethyl)-4-methylcoumarin, or 7-((2-methacrylamido) aminoethyl)-4-methylcoumarin; (b) N-(5-(2-azidoacetamido)pentyl)acrylamide (AzAPA); (c) N,N-dimethylacrylamide or N-isopropylacrylamide (NiPAM); and (d) acrylic acid (AAc) under conditions suitable to free-radical polymerization to form the hydrogel polymer having non-crosslinked copolymer chains, wherein at least some of the copolymer chains include a reactive coumarin end group from monomer (a); and
(2) crosslinking at least some of the copolymer chains by irradiating the hydrogel polymer with light having wavelength >270 nm, the crosslinking comprising dimers between the reactive coumarin end groups of the copolymer chains.

In various examples, the crosslinking comprises about 5 mole % of the available reactive coumarin end groups. In various examples, the hydrogel is in the physical form of nanogel particles.

In various examples, the free-radical polymerization comprises suspension/precipitation free-radical polymerization further comprising a free-radical initiator and a dispersant.

In various examples, a method for assembling a flow cell capable of sequencing nucleic acids comprises:
(a) treating a surface of the flow cell with any one of 3-mercaptopropylsilanetriol, 3-mercaptopropyltrimethoxysilane, or 3-mercaptopropyltriethoxysilane to form a surface having a plurality of reactive —SH groups tethered to the surface;
(b) reacting the plurality of —SH groups with an α,β-unsaturated carbonyl thiol-ene acceptor further comprising a reactive alkene or reactive 1,4-diene moiety capable of [2+2] or [2+2+2+2] photodimerization, respectively, at wavelengths >270 nm covalently tethered thereto to provide reactive alkene or reactive 1,4-diene groups on the surface;
(c) preparing a hydrogel polymer comprising copolymer chains further comprising a recurring unit of Formula (I) and a recurring unit of Formula (II);

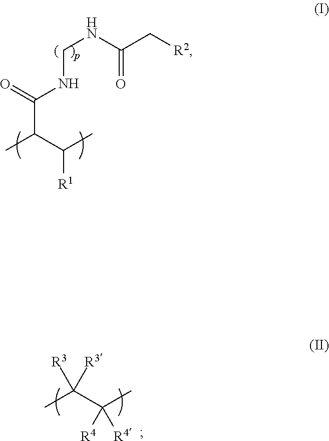

wherein:
R′ is H, alkyl, alkoxy, alkenyl, alkynyl, or optionally substituted variants thereof;
R² is —NH₂, alkyl, alkoxy, alkenyl, alkynyl, or optionally substituted variants thereof, or halogen, —N₃, —OH, —C(O)H, —NH═NH₂, —SCN, —CO₂H, —SH, glycidyl, epoxy, aziridine, triazoline;
p is an integer from 1 to 50;
each of R³, R^{3′}, R⁴, and R^{4′} is independently selected from —H, —R⁵, —OR⁵, —CO₂R⁵, —C(O)R⁵, —OC(O)R⁵, —C(O)NR⁶R⁷, —NR⁶R⁷ or a substructure of Formula (III),

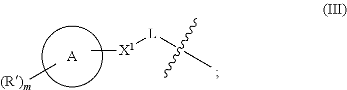

R⁵ is —H, —OH, alkyl, cycloalkyl, hydroxyalkyl, aryl, heteroaryl, or heterocyclyl;
each of R⁶ and R⁷ is independently selected from —H and alkyl;
A is an aryl or a thymidinyl moiety;
R′ is —H, alkyl, alkoxy, alkenyl, alkynyl, aryl, heterocyclyl or optionally substituted variants thereof, or halogen, —N₃, —OH, —C(O)H, —NH═NH₂, —SCN, —CO₂H, —SH, glycidyl, epoxy, or two R′ groups, when bonded to adjacent atoms on ring A and taken together with ring A, form a coumarinyl, an anthracenyl, an acenaphthylenyl, a thianaphthenyl-1-oxide or a thianaphthenyl -1,1-dioxide moiety;
X¹ is a bond, —(CH₂)_{q}—, —O— or —NH—;
L is a bivalent linker having a structure —(CH₂)_{q}—X²—C(═O)— or —(CH₂CH₂O)_{q}—X²—C(═O)—;
X² is —O— or —NH—;
m is an integer from 1 to 9; and
q is an integer from 0 to 50;
wherein at least some of the copolymer chains include at least one reactive alkene or reactive 1,4-diene end group capable of [2+2] or [2+2+2+2] photodimerization, respectively, at wavelengths >270 nm; and wherein at least some of the copolymer chains include at least one —N₃ or —C≡CH end group; and
(d) binding the hydrogel polymer onto the surface of the flow cell by (i) contacting the surface with the hydrogel polymer and (ii) irradiating the hydrogel polymer and the surface of the flow cell with incident light of wavelength >270 nm to form [2+2] or [2+2+2+2] photo-dimers between the reactive alkene or 1,4-diene groups on the surface and the reactive alkene or 1,4-diene end groups on respective copolymer chains.

In various examples, the irradiation step in (d) also crosslinks copolymer chains in the hydrogel polymer through [2+2] or [2+2+2+2] photoaddition of reactive alkene or reactive 1,4-diene end groups present on respective copolymer chains.

In various examples, the method further comprises grafting amplification primers onto the hydrogel polymer either before step (d) or after step (d) by performing click-chemistry reactions between a terminal alkyne substituent on the amplification primer and an azide end group on a respective copolymer chain or performing click-chemistry reactions between a terminal azide substituent on the amplification primer and an alkyne end group on a respective copolymer chain.

In various examples, the hydrogel polymer is prepared by subjecting a monomer mixture comprising (a) 7-((2-acryloyloxy)ethoxy)-4-methylcoumarin, 7-((2-methacryloyloxy)ethoxy)-4-methylcoumarin, 7-((2-acrylamido)ethoxy)-4-methylcoumarin, 7-((2-methacrylamido)ethoxy)-4-methylcoumarin, 7-((2-acryloyloxy)aminoethyl)-4-methylcoumarin, 7-((2-methacryloyloxy) aminoethyl)-4-methylcoumarin, 7-((2-acrylamido) aminoethyl)-4-methylcoumarin, or 7-((2-methacrylamido) aminoethyl)-4-methylcoumarin; (b) N-(5-(2-azidoacetamido)pentyl)acrylamide (AzAPA); (c) N,N-dimethylacrylamide or N-isopropylacrylamide (NiPAM); and (d) acrylic acid (AAc) to free-radical polymerization, thus forming copolymer chains, wherein the reactive alkene end groups capable of [2+2] photodimerization at wavelengths >270 nm comprise coumarin groups.

In various examples, the method further includes a recycling of the flow cell comprising removing the hydrogel polymer from the surface of the flow cell by irradiating the hydrogel polymer and the surface of the flow cell with light of wavelength <300 nm to reverse the dimerization and the binding of the hydrogel polymer to the surface of the flow cell.

In various examples, a method of synthesizing a hydrogel polymer comprises:
reacting a monomer mixture comprising (a) N-(5-(2-azidoacetamido)pentyl)acrylamide (AzAPA); (b) N,N-dimethylacrylamide or N-isopropylacrylamide (NiPAM); and (c) acrylic acid (AAc) under free-radical polymerization conditions to form a hydrogel polymer comprising copolymer chains having reactive azide end groups; and
reacting a least a portion of the azide end groups with N-(but-3-yn-1-yl)-2-(((2-oxo-2H-chromen -7-yl)oxy)methyl)acrylamide to form a hydrogel polymer having at least some copolymer chains with tethered 4-methylcoumarin end groups.

In various examples, the method further includes irradiating the hydrogel polymer with light of wavelength >270 nm, crosslinking at least some of the copolymer chains by coumarin photo-dimerization.

In various examples, the reacting is conducted inside a flow cell with the monomer mixture in contact with a surface of the flow cell. In various examples, the hydrogel polymer is in the physical form of nanogel particles.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF THE DRAWINGS

FIG. 1 schematically illustrates use of a photochemically-reversible hydrogel in a flow cell for nucleic acid sequencing in accordance with various examples of the present disclosure. FIG. 1 part A) illustrates attachment of a pre-formed polymer containing photo-switchable moieties to a functionalized flow cell surface. FIG. 1 part B) illustrates in situ formation of a hydrogel by polymerization of a monomer mixture on a flow cell surface.

FIG. 2 schematically illustrates attachment of photochemically-reversible nanogel particles onto a functionalized surface of a flow cell in accordance with various examples of the present disclosure, wherein the nanogel particles designated as RAP comprise the indicated functional groups for primer grafting and photo-reversibility to detach from the surface.

FIG. 3 schematically illustrates a partial chemical structure of a photochemically -reversible hydrogel or nanogel particle comprising poly(NDMAM-co-AzAPA-co-CAA) copolymer chains with molar ratio of NDMAM:AzAPA:CAA of 85:10:5 in accordance with various examples of the present disclosure. The coumarin groups, present as free end groups on the copolymer chains, are available to dimerize when irradiated with 365 nm light. The double reaction arrow indicates the crosslinking is cleaved when irradiated with 254 nm light.

FIG. 4 schematically illustrates two approaches for preparing cross-linkable hydrogels and nanogel particles in accordance with various examples of the present disclosure. In approach A, pre-formed polymer, such as PAZAM, is functionalized with N-(but-3-yn-1-yl)-2-(((2-oxo-2H -chromen-7-yl)oxy)methyl)acrylamide (“alkyne coumarin”) under copper catalyzed Blackpool grafting. In approach B, poly(NDMAM-co-AzAPA-co-CAA) copolymer chains are synthesized by free-radical polymerization of a monomer mixture comprising NDMAM, AzAPA, and CAA monomers.

FIG. 5A schematically illustrates functionalization of nanogel particles having available azide groups with N-(but-3-yn-1-yl)-2-(((2-oxo-2H-chromen-7-yl)oxy)methyl)acrylamide (“alkyne coumarin”) to form nanogel particles capable of [2+2] dimerization in accordance with various examples of the present disclosure.

FIG. 5B schematically illustrates synthesis of photochemically-reversible nanogel particles having poly(CAA-co-NiPAM-co-AzAPA-co-AAc-co-BisAM) copolymer chains by free-radical aqueous suspension/polymerization of a mixture of CAA, NiPAM, AzAPA, AAc and BisAM monomers in accordance with various examples of the present disclosure.

FIG. 6. illustrates a synthesis of the monomer 7-(acrylamido)-4-methylcoumarin (N-(4-methyl-2-oxo-2H-chromen-7-yl)acrylamide) by reaction of 7-amino-4-methylcoumarin and acryloyl chloride in dichloromethane (DCM) in accordance with various examples of the present disclosure.

FIG. 7 schematically illustrates functionalization of a flow cell surface in accordance with various examples of the present disclosure, wherein the surface is first silanized with 3-mercaptopropyltrimethoxysilane (MPTMS) followed by reaction of the tethered —SH groups with N-(2-((4-methyl-2-oxo-2H-chromen-7-yl)oxy)ethyl)acrylamide (“CAM”).

FIG. 8 schematically illustrates reactions of P5/P7 amplification primers having photo-reversible motifs in accordance with various examples of the present disclosure. In A) the photo-reversible motif is covalently bonded to the 5′-end of the primer. In B), photo-crosslinking induces primer grafting using a first wavelength (hυ1) followed by clustering and sequencing, after which the ssDNA can be removed by exposure to a second wavelength (hυ2) of incident radiation.

### DETAILED DESCRIPTION

The detailed description of examples herein makes reference to the accompanying drawings, which show examples by way of illustration and their best mode. While these examples are described in sufficient detail to enable those skilled in the art to practice the present subject matter, it should be understood that other examples may be realized and that logical, chemical, and mechanical changes may be made without departing from the spirit and scope of the subject matter provided herein. Thus, the detailed description is presented for purposes of illustration only and not of limitation. For example, unless otherwise noted, the steps recited in any of the method or process descriptions may be executed in any order and are not necessarily limited to the order presented. Furthermore, any reference to singular element or step includes plural element or step, and any reference to more than one component or step may include a singular element or step. Also, any reference to attached, fixed, connected or the like may include permanent, removable, temporary, partial, full and/or any other possible attachment option. Additionally, any reference to without contact (or similar phrases) may also include reduced contact or minimal contact.

### Terms

As used herein, the term “hydrogel” is intended to refer to a polymer comprising crosslinked or cross-linkable copolymer chains. Such polymers, or their monomer mixture precursors, can be coated on a surface either in continuous layers or in marked off regions. In various examples herein, a hydrogel polymer and a nanogel particle may have the same polymeric composition.

As used herein, the term “nanogel particle” is intended to refer to a nanoscale polymeric particle including copolymer chains that are optionally crosslinked. Simply for the sake of convenience, nanogel particles herein may be illustrated as “soccer balls,” i.e., substantially spherical in shape, although their structure might not be this simple. A spherical representation allows the reader to understand the concept of accessible functional groups in/on a nanogel particle since these groups, typically functional end groups on copolymer chains, can be shown to protrude from the surface of the particle. Nonetheless, particle size analysis can be performed, such as by light scattering, to obtain relevant particle size distributions or Z-averages. So even though nanogel particles herein may not be entirely spherical in shape with functional groups protruding from a surface, their average size can be determined. Typically, nanogel particles in accordance with the present disclosure have Z-averages of about 50 nm to about 500 nm. Also, recitations herein describe chemical reactions as taking place on a nanogel particle, at least for simplicity. Although nanogel particles herein are likely spherical in shape due to the method of synthesis comprising suspension/precipitation polymerization, the present disclosure is not limited in regard to particle shape. All nanogel “objects,” regardless of shape, are within the scope of the present disclosure. Further, it should be understood that since the particles comprise lightly crosslinked networks containing mostly water, various chemical reactions may take place both on and in a nanogel particle.

As used herein, the term “photochemically-reversible” is intended to refer to a property or characteristic of either hydrogel polymers or nanogel particles when the hydrogel polymer or the nanogel particles include at least some copolymer chains having at least one reactive alkene or reactive 1,4-diene end group capable of photochemically reversible [2+2] or [2+2+2+2] cycloaddition, i.e., photodimerization. Hydrogel polymers and nanogel particles having photochemical reversibility can be reversibly attached and removed from certain surfaces.

As used herein, the term “end group” is intended to refer to a substituent at a physically terminal position on a copolymer chain structure of a hydrogel polymer or nanogel particle, including positions on ends of a polyene backbone of a copolymer chain or at the end of branches appending from the polyene backbone. In particular, copolymers herein may be characterized as polyenes, but certain reactive end groups of interest (e.g., -coumarin, -4-methylcoumarin, —N₃, —CO2H, —C≡CH, etc.) may be bonded to the ends of appendages branched off the polyene backbone and thus remain sterically accessible for various chemical reactions.

As used herein, the term “dual functionality” is intended to refer to a property or characteristic of both hydrogels and nanogel particles when the hydrogel or nanogel particles include at least some copolymer chains having at least two types of functional substituents present as copolymer chain end groups, such as (1) at least one reactive alkene or reactive 1,4-diene end group capable of [2+2] or [2+2+2+2] photodimerization, respectively, at wavelengths >270 nm and (2) at least one of carboxylic acid end groups, —N₃ end groups, and/or —C≡CH end groups. The functional groups in type (2) can be used in specific binding or conjugation reactions. Dual functionality is intended to include “multiple functionalities” in instances where there are more than two types of reactive end groups on copolymer chains. For example, hydrogels and nanogel particles having dual functionality allow covalent attachment of amplification primers onto the hydrogel or nanogel particles (such as by reacting alkyne-functionalized amplification primers with free —N₃ end groups present on the copolymer chains of the hydrogel polymer or nanogel particles, or vice versa) and binding of dual functionalized hydrogels or nanogel particles to surfaces in a flow cell (such as by reacting free carboxylic acid end groups on the copolymer chains of the hydrogel or nanogel particles with functionalized groups appended to the flow cell surfaces). In other examples, the presence of copolymer chains having at least one reactive alkene or reactive 1,4-diene end group capable of [2+2] or [2+2+2+2] photodimerization, respectively, at wavelengths >270 nm, allows for binding of hydrogels or nanogel particles onto surfaces previously functionalized with reactive alkenes or 1,4-dienes, wherein the binding to the surface comprises photo-dimerization between reactive end groups on the copolymer chains and alkene or 1,4-diene groups tethered on the surface.

As used herein, the term “temperature responsiveness” is intended to refer to a property or characteristic of nanogel particles when the nanogel particles include at least some copolymer chains having sections of polymer structure physically responsive to temperature. More specifically, nanogel particles that are temperature responsive exhibit shrinking when exposed to increasing or decreasing temperatures, and exhibit swelling when exposed to the opposite temperature trend. In various examples, nanogel particles having copolymer chains with blocks of poly(NiPAM) shrink with increasing temperature. This temperature responsiveness provides methods for placing nanogel particles into holes, such as nano-wells, and then locking them in place simply by temperature manipulations. Although temperature responsiveness would be present to some degree in hydrogels comprising at least some copolymer chains having sections of polymer structure physically responsive to temperature, such as blocks of poly(NiPAM) in copolymer chains, temperature responsive of a polymeric layer may not be as useful of a characteristic as it proves to be in nanogel particles, since nanogel particles are physically manipulated on surfaces.

As used herein, the term “pH responsiveness” is intended to refer to a property or characteristic of hydrogels and nanogel particles when the hydrogel or nanogel particles include at least some copolymer chains having carboxylic acid end groups, such that at certain pH ranges these groups are predominantly —CO₂H and at other pH ranges these groups are predominantly —CO₂⁻. Stated another way, pH responsive carboxylic acid end groups on at least some of the copolymer chains of the hydrogel or nanogel particles impart pH responsiveness to the hydrogel or nanogel particles. In various examples, the pH responsiveness allows for pH-driven binding of hydrogel or nanogel particles to functionalized flow cell surfaces.

As used herein, the term “dual stimuli (temperature/pH)” is intended to refer to the combination of temperature responsiveness and pH responsiveness properties (per the above definitions) exhibited by certain hydrogels and nanogel particles. In various examples, blocks of poly-NiPAM in copolymer chains of a hydrogel or nanogel particle imparts temperature responsiveness to the hydrogel or nanogel particles (i.e., shrinking/swelling), whereas the presence of AAc units in copolymer chains of a hydrogel or a nanogel particle contributes to the pH responsiveness of the hydrogel or nanogel particles.

As used herein, the term “suspension/precipitation polymerization” is intended to refer to a free-radical suspension polymerization reaction in which water-soluble monomers and a free-radical initiator produce the polymeric nanogel particles as a dispersed solid phase when using a dispersant or steric stabilizer and vigorous stirring of the reaction mixture. Suspension/precipitation polymerization is thoroughly explained in the academic reference, S. Beck, et al., Chapter 3, pp 21-85 in “Polymer Science and Nanotechnology-Fundamentals and Applications,” Elsevier, 2020, https://doi.org/10.1016/B978-0-12-816806-6.00003-0, the entire contents of which are incorporated by reference herein. Further, the present disclosure is not limited to this particular polymerization method for the synthesis of nanogel particles. For example, emulsion polymerization techniques may be employed, and non-aqueous solvents may be used.

As used herein, the acronym “CAA” is intended to refer to the monomer, 2-((4-methyl -2-oxo-2H-chromen-7-yl)oxy)ethyl acrylate, (or more simply, “coumarin acrylate”), having the chemical structure,

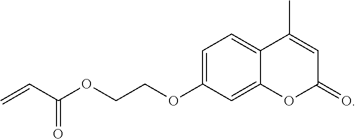 [SMILES: C=CC(=O)OCCOc1ccc2c(C)cc(=O)oc2c1]

As used herein, the acronym “CAM” is intended to refer to the monomer, N-(2-((4-methyl-2-oxo-2H-chromen-7-yl)oxy)ethyl)acrylamide, (or more simply, “coumarin acrylamide”), having the chemical structure,

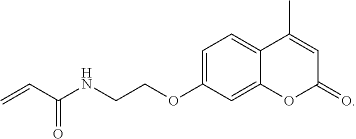 [SMILES: C=CC(=O)NCCOc1ccc2c(C)cc(=O)oc2c1]

As used herein, the acronym “AzAPA” is intended to refer to the monomer, N-(5-(2-azidoacetamido)pentyl)acrylamide.

As used herein, the acronym “NiPAM” is intended to refer to the monomer, N-isopropylacrylamide.

As used herein, the acronym “NDMAM” is intended to refer to the monomer, N,N-dimethylacrylamide.

As used herein, the acronym “BisAM” is intended to refer to the multifunctional monomer, N,N′-methylenebisacrylamide.

As used herein, the acronym “PAG” is intended to refer to the monomer, propargyl acrylate.

As used herein, the acronym “PAM” is intended to refer to the monomer, N-propargyl acrylamide.

As used herein, the acronym “AAc” is intended to refer to the monomer, acrylic acid.

As used herein, the acronym “BrAPA” is intended to refer to the monomer N-(5-(2-bromoacedamido)pentyl)acrylamide, used in various examples to form PAZAM coatings on flow cell (FC) surfaces.

As used herein, the term “alkyne coumarin” is intended to refer to the compound and monomer, N-(but-3-yn-1-yl)-2-(((2-oxo-2H-chromen-7-yl)oxy)methyl)acrylamide, having the chemical structure,

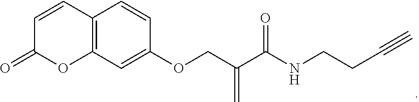 [SMILES: C#CCCNC(=O)C(=C)COc1ccc2ccc(=O)oc2c1]

As used herein, the acronym “SDS” is intended to refer to the anionic dispersant sodium dodecyl sulfate.

As used herein, the acronym “APS” is intended to refer to the free-radical polymerization initiator ammonium persulfate.

As used herein, the acronym “ANA” is intended to refer to hydrogel or nanogel particles including poly(AzAPA-co-NiPAM-co-AAc-co-BisAM) copolymer chains. ANA hydrogels and nanogel particles feature both carboxylic acid and —N₃ end groups on at least some copolymer chains.

As used herein, the acronym “PANA” is intended to refer to hydrogel and nanogel particles including poly(PAG-co-NiPAM-co-AAc-co-BisAM) copolymer chains. PANA hydrogels and particles feature both carboxylic acid and —C≡CH end groups on at least some copolymer chains.

As used herein, the acronym “PANA'” is intended to refer to hydrogel and nanogel particles including poly(PAM-co-NiPAM-co-AAc-co-BisAM) copolymer chains. PANA′ hydrogels and particles feature both carboxylic acid and —C≡CH end groups on at least some copolymer chains.

As used herein, the term “flow cell” (and acronym “FC”) is intended to refer to a vessel having a chamber (e.g., a flow channel or “lane”) where a reaction can be carried out, an inlet for delivering reagent(s) to the chamber, and an outlet for removing reagent(s) from the chamber. In various examples, the chamber enables the detection of the reaction that occurred in the chamber. For example, the chamber can include one or more transparent surfaces allowing for the optical detection of arrays, optically labeled molecules, or the like, in the chamber. In various examples, polymeric materials such as nanogel particles or hydrogel polymer coatings may be attached to surfaces in a flow cell channel.

As used herein, the term “covalently attached” or “covalently bonded” is intended to refer to the forming of a chemical bonding that is characterized by the sharing of pairs of electrons between atoms. For example, a covalently attached polymer coating is intended to refer to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

As used herein, the acronym “PAZAM” is intended to refer to a functionalized polymeric coating including poly(N-(5-azidoacetamidylpentyl) acrylamide-co-acrylamide.

As used herein, the acronym “DMTMM” is intended to refer to the compound, 4-(4,6-dimethoxy-1,3,5-triazin-2-yl)-4-methylmorpholinium chloride.

As used herein, the acronym “CuAAC” is intended to refer to copper-catalyzed azide-alkyne cycloaddition click-chemistry.

As used herein, the acronym “dz” or “Dz” is intended to refer to a “Z-average” reported from a particle size analysis and is known in the art as a reliable measure of the average size of a particle size distribution. A Z-average can be directly ascertained from a light-scattering experiment using a nanoparticle analyzer. See, for example, J. C. Thomas, “The determination of log normal particle size distributions by dynamic light scattering,” J. Colloid Interface Sci., 117(1), 187-192 (1987).

As used herein, the acronym “SBS” is intended to refer to “Sequencing by Synthesis,” a sequencing technology using fluorescently labeled nucleotides to sequence multitudes of clusters present on a flow cell surface in parallel. In some examples of SBS, during each sequencing cycle, a single labeled dNTP is added to the nucleic acid chain. The nucleotide label serves as a terminator for polymerization, such that after each dNTP incorporation, the fluorescent dye is imaged to identify the base and then enzymatically cleaved to allow incorporation of the next nucleotide. Further understanding of SBS is disclosed in PCT Application Publications WO2018/119101 and WO2020/005501 (both to Illumina, Inc.), the disclosures of which are incorporated herein by reference in their entireties.

As used herein, the term “seeding” is intended to refer to binding of a single stranded oligonucleotide (ssDNA) to an amplification primer covalently attached to a nanogel particle. In various examples, seeding includes monoclonal seeding.

As used herein, the term “particle clustering” is intended to refer to clustering of multiple copies of one type (monoclonal) or multiple types (polyclonal) of a sequencing template or templates, respectively, on a single nanogel particle previously grafted with amplification primers and having seeded ssDNA. The term particle clustering is intended to refer to activity on a nanogel particle and is not to be confused with physical clustering of nanogel particles themselves.

As used herein, the term “suspension clustering” is intended to refer to a process whereby nanogel particles, previously seeded with a ssDNA and clustered, are subsequently captured on a FC for sequencing.

As used herein, the term “on-board clustering” is intended to refer to a process whereby nanogel particles of suitable size (e.g., ranging from about 200 nm to about 400 nm), previously grafted with primer density compatible with sequencing and subsequently captured in nano-wells of a FC (e.g., HiSeqX™ platform), are then clustered to generate enough copy of a template usable for sequencing.

As used herein, the term “Typhoon” is intended to refer to the Amersham™ Typhoon™ a commercially available laser-scanner platform from Cytiva Life Sciences, for imaging and quantitation of nucleic acids and proteins. When used as an action verb, the term is intended to refer to performing a method of imaging, such as fluorescent imaging, using the Amersham™ Typhoon™ laser-scanner.

For additional acronyms and terminology relating to hydrogel coatings in flow cells and the use of these flow cells in SBS, see U.S. Pat. No. 10,919,033 (Illumina, Inc.), the disclosure of which is incorporated herein in its entirety.

As used herein, any “R” group designated on a chemical structure, such as, R¹, R², R³, R⁴, R⁵, R⁶, R⁷, and R⁸ and so forth, represent substituents in organic chemistry that can be attached to the indicated atom to which the “R” group is bonded. An R group may be substituted or unsubstituted. If two “R” groups are described as being “taken together” to form a cyclic structure, the R groups and the atoms to which they are attached can form a cycloalkyl, aryl, heteroaryl, or heterocyclic ring. In some instances, the ring thus formed may create a bicyclic or tricyclic structure.

As used herein, the term “alkyl” is intended to refer to linear or branched monovalent fully saturated hydrocarbon substituents, optionally substituted with one or more functional groups anywhere on the substituent. Unless otherwise specified, an alkyl group may contain any number of carbon atoms, such as for example, C₁-C₂₄, C₁-C₁₈, C₁-C₁₀, C₁-C₈, C₁-C₆, or C₁-C₄. Examples of alkyl substituents include, but are not limited to, methyl, ethyl, n-propyl, isopropyl, n-butyl, isobutyl, sec-butyl, tert-butyl, n-pentyl, iso-pentyl neo-pentyl, n-hexyl, iso-hexyl, octadecyl, dodecyl, and so forth. An alkyl substituent herein may be substituted, i.e., having one or more substituent groups appended on the alkyl group or incorporated within the alkyl chain. A substitution within an alkyl substituent chain may include an ether, sulfide, or imine linkage, i.e., —O—, —S—, or —N═, for example, or some other intervening heteroatom(s). Examples of substitution on an alkyl substituent include, but are not limited to, —CN, —N₃, —NH₂, —NHR, —N(R)₂, —N(R)₃⁺, —NO₂, —NH—NH₂, —NH—NHR, —NH—NR₂, -halo, —SH, —SR, —S(═O)R, —SO₂R, —OPO₃²⁻, —PO₃²⁻, —OH, —OR, —C(═O)R, —OC(═O)R, —CO₂R, —NHC(═O)R, —NRC(═O)R, —C(═O)NHR, —C(═O)NR₂, alkyl, alkenyl, cycloalkyl, heterocyclyl, and aryl, wherein each R above is independently selected from hydrogen -H and an alkyl moiety, including, for example, C₁₋₆ alkyl (e.g., —CH₃, —C₂H₅, -isopropyl, -tert-butyl, etc.), C₁₋₆ alkoxy (e.g., —OCH₃, —OC₂H₅), halogenated C₁₋₆ alkyl (e.g., —CF₃, —CHF₂, —CH₂F), and halogenated C₁₋₆ alkoxy (e.g., —OCF₃, —OC₂F₅).

As used herein, the term “cycloalkyl” includes any 3-, 4-, 5-, 6-, 7-, or 8-membered, saturated or unsaturated, non-aromatic carbocyclic ring, optionally substituted with one or more functional groups at any location on the cyclic substituent. Examples of cycloalkyl groups include, but are not limited to, cyclopropyl, cyclobutyl, cyclopentyl, 1-, 2-, or 5-cyclopentadienyl, cyclohexyl, 1-, 3- or 4-cyclohexenyl, 1-, 2-, or 5-(1,3-cyclohexadienyl), 1- or 3-(1,4-cyclohexadienyl), cycloheptyl, 1-, 3-, 4-, or 5-cycloheptenyl, cyclooctanyl, and so forth. Examples of substitution on an cycloalkyl substituent include, but are not limited to, —CN, —N₃, —NH₂, —NHR, —N(R)₂, —N(R)₃⁺, —NO₂, —NH—NH₂, —NH—NHR, —NH—NR₂, -halo, —SH, —SR, —S(═O)R, —SO₂R, —OPO₃²⁻, —PO₃²⁻, —OH, —OR, —C(═O)R, —OC(═O)R, —CO₂R, —NHC(═O)R, —NRC(═O)R, —C(═O)NHR, —C(═O)NR₂, alkyl, alkenyl, cycloalkyl, heterocyclyl, and aryl, wherein each R above is independently selected from —H and an alkyl moiety, including, for example, C₁₋₆ alkyl (e.g., —CH₃, —C₂H₅, -isopropyl, -tert-butyl, etc.), C₁₋₆ alkoxy (e.g., —OCH₃, —OC₂H₅), halogenated C₁₋₆ alkyl (e.g., —CF₃, —CHF₂, —CH₂F), and halogenated C₁₋₆ alkoxy (e.g., —OCF₃, —OC₂F₅).

As used herein, the term “alkenyl” is intended to refer to linear or branched monovalent or divalent unsaturated hydrocarbon substituents, optionally substituted with one or more functional groups anywhere on or within the substituent. An alkenyl substituent can be viewed as being divalent if the sp² carbon is part of a molecule bearing the alkenyl substituent. An illustrative example is methylenecyclohexane, which can be viewed as cyclohexane substituted with a methylene group (i.e., a divalent alkenyl substituent, ═CH₂). Unless otherwise specified, an alkenyl group may contain any number of carbon atoms, such as for example, C₁-C₂₄, C₁-C₁₈, C₁-C₁₀, C₁-C₈, or C₁-C₆, and any degrees of unsaturation. Examples of alkenyl substituents include, but are not limited to, methylene/methylidine (═CH₂), ethylene/ethenyl (—CH═CH₂ or ═CH—CH₃), propylene/propenyl (—CH₂—CH═CH₂, cis or trans —CH═CH—CH₃, ═C(CH₃)₂, or cis or trans ═CH—CH₂CH₃), and so forth. An alkenyl substituent herein may be substituted, i.e having one or more substituent groups appended on the alkenyl group or incorporated within the alkenyl chain. A substitution within the alkenyl substituent may include an ether, sulfide, or imine linkage, i.e., —O—, —S—, or —N═, for example, or some other intervening heteroatom(s). Examples of substitution on an alkenyl substituent include, but are not limited to, —CN, —N₃, —NH₂, —NHR, —N(R)₂, —N(R)₃⁺, —NO₂, —NH—NH₂, —NH—NHR, —NH—NR₂, -halo, —SH, —SR, —S(═O)R, —SO₂R, —OPO₃²⁻, —PO₃²⁻, —OH, —OR, —C(═O)R, —OC(═O)R, —CO₂R, —NHC(═O)R, —NRC(═O)R, —C(═O)NHR, —C(═O)NR₂, alkyl, alkenyl, cycloalkyl, heterocyclyl, and aryl, wherein each R above is independently selected from an alkyl moiety, including, for example, C₁₋₆ alkyl (e.g., —CH₃, —C₂H₅, -isopropyl, -tert-butyl, etc.), C₁₋₆ alkoxy (e.g., —OCH₃, —OC₂H₅), halogenated C₁₋₆ alkyl (e.g., —CF₃, —CHF₂, —CH₂F), and halogenated C₁₋₆ alkoxy (e.g., —OCF₃, —OC₂F₅).

As used herein, the term “aryl” includes any aromatic ring or fused polycyclic aromatic ring system, such as phenyl, naphthyl, anthracenyl, and phenanthrenyl, optionally substituted with one or more functional groups anywhere on the aromatic substituent. An unsubstituted phenyl substituent may also be denoted as —C₆H₅ or more simply, —Ph. Aromatic heterocyclic rings and fused ring heterocyclic aromatic substituents are distinct and are included in the definition of heterocycyl substituents set forth below. Examples of substitution on an aryl substituent include, but are not limited to, —CN, —N₃, —NH₂, —NHR, —N(R)₂, —N(R)₃⁺, —NO₂, —NH—NH₂, —NH—NHR, —NH——NR₂, -halo, —SH, —SR, —S(═O)R, —SO₂R, —OPO₃²⁻, —PO₃²⁻, —OH, —OR, —C(═O)R, —OC(═O)R, —CO₂R, —NHC(═O)R, —NRC(═O)R, —C(═O)NHR, —C(═O)NR₂, alkyl, alkenyl, cycloalkyl, heterocyclyl, and aryl, wherein each R above is independently selected from an alkyl moiety, including, for example, C₁₋₆ alkyl (e.g., —CH₃, —C₂H₅, -isopropyl, -tert-butyl, etc.), C₁₋₆ alkoxy (e.g., —OCH₃, —OC₂H₅), halogenated C₁₋₆ alkyl (e.g., -—CF₃, —CHF₂, —CH₂F), and halogenated C₁₋₆ alkoxy (e.g., —OCF₃, —OC₂F₅).

As used herein, “heterocycle” is intended to refer to an unsubstituted or optionally substituted, saturated, unsaturated or aromatic, carbocyclic ring interrupted in its carbocyclic structure by at least one heteroatom selected from oxygen (O), sulfur (S) or nitrogen (N). As used herein, the term “heterocyclyl” is intended to refer to a heterocycle as a substituent group, being attached to another atom in a compound from any C atom or heteroatom present in the heterocyclic ring. For example, “pyridinyl” includes 2-, 3- and 4-pyridinyl moieties as substituent groups. Heterocycles may be monocyclic or fused polycyclic in structure. Examples of optional substitution on a heterocycyl substituent include, but are not limited to, oxo, —CN, —N₃, —NH₂, NHR, —N(R)₂, —N(R)₃⁺, —NO₂, —NH—NH₂, —NH—NHR, —NH—NR₂, -halo, —SH, —SR, —S(═O)R, —SO₂R, —OPO₃²⁻, —PO₃²⁻, —OH, —OR, —C(═O)R, —OC(═O)R, —CO₂R, —NHC(═O)R, —NRC(═O)R, —C(═O)NHR, —C(═O)NR₂, alkyl, alkenyl, cycloalkyl, heterocyclyl, and aryl, wherein each R above is independently selected from an alkyl moiety, including, for example, C₁₋₆ alkyl (e.g., —CH₃, —C₂H₅, -isopropyl, -tert-butyl, etc.), C₁₋₆ alkoxy (e.g., —OCH₃, —OC₂H₅), halogenated C₁₋₆ alkyl (e.g., —CF₃, —CHF₂, —CH₂F), and halogenated C₁₋₆ alkoxy (e.g., —OCF₃, —OC₂F₅).

Examples of heterocycles include but are not limited to: azepinyl, aziridinyl, azetyl, azetidinyl, coumarinyl (2H-chromen-2-one), diazepinyl, dithiadiazinyl, dioxazepinyl, dioxolanyl, dithiazolyl, furanyl, isooxazolyl, isothiazolyl, imidazolyl, morpholinyl, morpholino, oxetanyl, oxadiazolyl, oxiranyl, oxazinyl, oxazolyl, piperazinyl, pyrazinyl, pyridazinyl, pyrimidinyl, piperidyl, piperidino, pyridyl, pyranyl, pyrazolyl, pyrrolyl, pyrrolidinyl, thiatriazolyl, tetrazolyl, thiadiazolyl, triazolyl, thiazolyl, thienyl, tetrazinyl, thiadiazinyl, triazinyl, thiazinyl, thiopyranyl furoisoxazolyl, imidazothiazolyl, thienoisothiazolyl, thienothiazolyl, imidazopyrazolyl, cyclopentapyrazolyl, pyrrolopyrrolyl, thienothienyl, thiadiazolopyrimidinyl, thiazolothiazinyl, thiazolopyrimidinyl, thiazolopyridinyl, oxazolopyrimidinyl, oxazolopyridyl, benzoxazolyl, benzisothiazolyl, benzothiazolyl, imidazopyrazinyl, purinyl, pyrazolopyrimidinyl, imidazopyridinyl, benzimidazolyl, indazolyl, benzoxathiolyl, benzodioxolyl, benzodithiolyl, indolizinyl, indolinyl, isoindolinyl, furopyrimidinyl, furopyridyl, benzofuranyl, isobenzofuranyl, thienopyrimidinyl, thienapyridyl, benzothienyl, cyclopentaoxazinyl, cyclopentafuranyl, benzoxazinyl, benzothiazinyl, quinazolinyl, naphthyridinyl, quinolinyl, isoquinolinyl, benzopyranyl, pyridopyridazinyl and pyridopyrimidinyl. Further examples of heterocyclic systems may be found in A. Katritzky, et al., Handbook of Heterocyclic Chemistry, 3^{rd} Ed., Elsevier, 2010, the entire contents of which are incorporated by reference herein.

### General Examples

In various examples of the present disclosure, novel photochemically-reversible hydrogel polymers and polymeric nanogel particles are described. Various nanogel particles herein exhibit dual functionality through the presence of at least two types of reactive end groups on copolymer chains within the nanogel particles. For example, the present nanogel particles may exhibit temperature responsiveness wherein nanogel particles can shrink or swell in response to temperature changes, and pH responsiveness that assists in surface binding reactions. Nanogel particles in accordance with the present disclosure may, among other things, find use in nucleic acid sequencing methods, in particular within flow cells used in SBS methods.

In various examples, nanogel particles are prepared by a suspension/precipitation free radical polymerization of various monomer types. Nanogel particles herein are described by the synthetic processes used to prepare them, i.e., the monomers used in the suspension/precipitation free radical polymerization reaction and the reaction conditions, and also structurally, such as by describing certain recurring units present in copolymer chains of the nanogel particles thus prepared, along with physical properties. In various examples, recurring monomer units in a copolymer chain of a nanogel particle may include part of a block within a block copolymer.

In various examples, photochemically-reversible hydrogel polymers and nanogel particles include copolymer chains that can be crosslinked and/or are at least partially crosslinked. Crosslinking is expected, for example, if a multifunctional monomer is used in the suspension/precipitation free radical polymerization along with other monomer types. As explained herein, crosslinking may also be initiated photochemically, such as to dimerize reactive alkene or diene end groups on copolymer chains.

In various examples, incorporating monomers resulting in temperature or pH responsive nanogel particles, the nanogel particle sizes can be fine-tuned to be adaptable with any step in SBS sequencing protocol, such as, library seeding, nanogel particle capture in nano-wells of a FC, clustering on particles, and sequencing on particles. In various examples, temperature responsiveness can be incorporated using LCST (lower critical solution temperature) or UCST (upper critical solution temperature).

### Goals and General Considerations

In various examples, photo-switchable chemistry for photochemically-reversible hydrogels and nanogel particles provides a potential solution for sustainability by enabling sequencing flow cells to be reusable.

In various examples, photochemically-reversible hydrogel or nanogel particles are attached to a functionalized FC surface, followed by the clustering and sequencing steps. Once this is completed, the photochemically-reversible hydrogel or nanogel particles is/are cleaved from the surface and discarded from the FC lane by flushing. A fresh solution of photochemically-reversible hydrogels or nanogel particles can be then used for the next sequencing steps.

Many physical, chemical, and photo-assisted switchable/reversible pathways have been proposed for these purposes. One advantage of photo-reversible chemistries compared to other systems is that additional chemical or physical stimuli are not required. Many photo-reversible crosslinking chemistries have been explored which can be activated by a wide of wavelengths. They are categorially divided as short wavelengths (<400 nm) and long wavelengths (400-1000 nm). However, they are often activated and deactivated using long wavelength (600-1000 nm). In principle, these reversible chemistries should also demonstrate zero (or low) absorbance at wavelengths longer than 400 nm in order to be compatible with SBS sequencing methods (e.g., as marketed by Illumina). By using short wavelengths for photo-cleavage and photo-crosslinking, respectively, derivatives of coumarin, anthracene, thymine, cinnamic acid and stilbene are good candidates for photochemically-reversible hydrogels and nanogel particles used in nucleic acid sequencing methods.

In various examples, photochemically-reversible hydrogels and nanogel particles comprise copolymer chains having either reactive alkene or reactive 1,4-diene end groups that can participate in [2+2] or [2+2+2+2] photoaddition reactions, respectively. These photoaddition reactions may be characterized as photodimerization, usable to reversibly attach hydrogels or nanogel particles to functionalized FC surfaces and/or to crosslink copolymer chains.

In various examples, TABLE 1 provides a summary of short wavelength photo-switchable chemistries incorporated in photochemically-reversible hydrogels and nanogel particles herein.

TABLE 1
Photo-switchable (short wavelength) chemistries

| Photo-reactive moiety present | | |
|---|---|---|
| in hydrogels and | | |
| nanogel particles of | Photo-crosslinking | Photo-cleaving |
| the present disclosure | condition (hυ1) | condition (hυ2) |
| Coumarin | UV, visible >320 nm | UV < 280 nm |
| Thymine | UV, >270 nm | UV < 249 nm |
| Cinnamic | UV, >300 nm | UV < 260 nm |
| Stilbene | UV, visible >300 nm | UV < 280 nm |
| Anthracene | UV, visible >350 nm | UV < 300 nm |

### Monomers for Photochemically-Reversible Hydrogel Polymers and Nanogel Particle Synthesis

In various examples, a first type of monomer for use in synthesizing photochemically-reversible hydrogel polymers and polymeric nanogel particles in a free radical polymerization reaction include species having a structure:

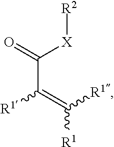

wherein:
each of R¹, R^{1′}, and R^{1″} is independently selected from H, halogen, alkyl, alkoxy, alkenyl, alkynyl, cycloalkyl, aryl, heteroaryl, or heterocyclyl;
X is —O— or —NH—; and
R² is —CH₂—C≡CH, or R² has a structure:

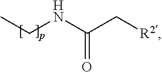

wherein R^{2′} is —NH₂, alkyl, alkoxy, alkenyl, alkynyl, or optionally substituted variants thereof, or halogen, —N₃, —OH, —C(O)H, —NH═NH₂, —SCN, —CO₂H, —SH, glycidyl, epoxy, aziridine, triazoline, or —C≡CH; and
p is an integer of 1 to 50.

Examples of this first type of monomer include, but are not limited to, propargyl acrylate, N-propargyl acrylamide, N-(5-(2-azidoacetamido)pentyl)acrylamide, (2-methacryloyloxy)trimethylammonium chloride, 2-acrylamido-2-methyl-1-propanesulfonic acid, [2-(acryloyloxy)ethyl]trimethylammonium chloride, and 2-hydroxyethylmethacrylate.

In various examples, a second type of monomer for use in synthesizing photochemically-reversible hydrogel polymers and polymeric nanogel particles in a free radical polymerization reaction include species having a structure:

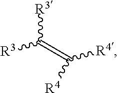

wherein:
each of R³, R^{3′}, R⁴, and R^{4′} is independently selected from —H, —R⁵, —OR⁵, —CO₂R⁵, —C(O)R⁵, —OC(O)R⁵, —C(O)NR⁶R⁷, —NR⁶R⁷ or a substructure of Formula (III),

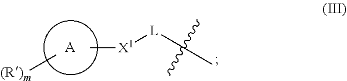

R⁵ is —H, —OH, alkyl, cycloalkyl, hydroxyalkyl, aryl, heteroaryl, or heterocyclyl;
each of R⁶ and R⁷ is independently selected from —H and alkyl;
A is an aryl or a thymidinyl moiety;
R′ is —H, alkyl, alkoxy, alkenyl, alkynyl, aryl, heterocyclyl or optionally substituted variants
thereof, or halogen, —N₃, —OH, —C(O)H, —NH═NH₂, —SCN, —CO₂H, —SH, glycidyl, epoxy, or two R′ groups, when bonded to adjacent atoms on ring A and taken together with ring A, form a coumarinyl, an anthracenyl, an acenaphthylenyl, a thianaphthenyl-1-oxide or a thianaphthenyl -1,1-dioxide moiety;
X¹ is a bond, —(CH₂)_{q}—, —O— or —NH—;
L is a bivalent linker having a structure —(CH₂)_{q}—X²—C(═O)— or —(CH₂CH₂O)_{q}—X²—C(═O)—;
X² is —O— or —NH—;
m is an integer from 1 to 9; and
q is an integer from 0 to 50.

In various examples of monomers of the second type, Formula (III) has the subgenus structure (IV):

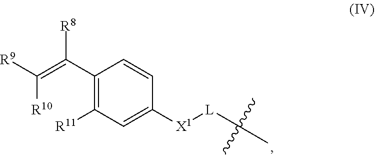

wherein each of R⁸ and R⁹ is —H, alkyl, cycloalkyl, hydroxyalkyl, aryl, heteroaryl, or heterocyclyl; each R¹⁰ and R¹¹ is independently —H, alkyl, cycloalkyl, hydroxyalkyl, aryl, heteroaryl, or heterocyclyl, or R¹⁰ is —C(═O)—, R¹¹ is —O—, and R¹⁰ and R¹¹ are bonded together such that Formula (IV) comprises a substituted coumarin moiety.

Examples of this second type of monomer include, but are not limited to, acrylic acid, methacrylic acid, acrylamide, methacrylamide, N-isopropylacrylamide, N-isopropylmethacrylamide, N,N-dimethylacrylamide, N,N-dimethylmethacrylamide, N-vinylpyrrolidone, N-vinylpyridine, N-(4-methyl-2-oxo-2H-chromen-7-yl)acrylamide, 4-methyl -2-oxo-2H-chromen-7-yl acrylate, 2-((4-methyl-2-oxo-2H-chromen-7-yl)oxy)ethyl acrylate, N-(2-((4-methyl-2-oxo-2H-chromen-7-yl)oxy)ethyl)acrylamide, 2-((4-methyl-2-oxo-2H-chromen-7-yl)amino)ethyl acrylate, N-(2-((4-methyl-2-oxo-2H-chromen-7-yl)amino)ethyl)acrylamide, N-(but-3-yn-1-yl)-2-(((2-oxo-2H-chromen-7-yl)oxy)methyl)acrylamide, 2-(5-methyl-2,6-dioxo-3,6-dihydropyrimidin-1(2H)-yl)ethyl acrylate, N-(2-(5-methyl-2,6-dioxo-3,6-dihydropyrimidin -1(2H)-yl)ethyl)acrylamide, 2-(anthracen-2-yloxy)ethyl acrylate, N-(2-(anthracen-2-yloxy)ethyl)acrylamide, 2-(anthracen-2-ylamino)ethyl acrylate, N-(2-(anthracen-2-ylamino)ethyl)acrylamide, (E)-2-(4-(2-(quinoxalin-2-yl)vinyl)phenoxy)ethyl acrylate, (E)-N-(2-(4-(2-(quinoxalin-2-yl)vinyl)phenoxy)ethyl)acrylamide, (E)-2-((4-(2-(quinoxalin-2-yl)vinyl)phenyl)amino)ethyl acrylate, and (E)-N-(2-((4-(2-(quinoxalin-2-yl)vinyl)phenyl)amino)ethyl)acrylamide.

In various examples, photochemically-reversible hydrogel polymers are prepared under various free radical polymerization reaction conditions by reacting at least one first type of monomer and at least one second type of monomer, in accordance with the above recited structures. For a review of synthesis methods, see, for example, U. Madduma-Bandarage, et al., “Synthetic Hydrogels: Synthesis, Novel Trends, and Applications,” J. Appl. Polym. Sci., 2021; 138: e50376, https://doi.org/10.1002/app.50376, and E. Ahmed, “Hydrogel: Preparation, Characterization, and Applications: A Review,” J. Adv. Res., 6(2), 105-121 (2015), the entire contents of each of which are incorporated by reference herein.

With these two types of monomers used in a free radical polymerization reaction, various photochemically-reversible hydrogel polymers resulting therefrom include copolymer chains having at least a first repeating unit incorporating the first type of monomer and at least a second repeating unit incorporating the second type of monomer.

In various examples, photochemically-reversible nanogel particles are prepared under suspension/precipitation or emulsion free radical polymerization reaction conditions by reacting at least one first type of monomer and at least one second type of monomer, in accordance with the above recited structures. With these two types of monomers used in a suspension/precipitation or emulsion free radical polymerization reaction, resulting photochemically-reversible nanogel particles include copolymer chains having at least a first repeating unit incorporating the first type of monomer and at least a second repeating unit incorporating the second type of monomer.

Multifunctional monomers that can be included in a free radical polymerization reaction to form photochemically-reversible hydrogel polymers and nanogel particles having some degree of crosslinking between copolymer chains include, but are not limited to, N,N′-methylenebisacrylamide, N,N′-methylenebismethacrylamide, polyethylene glycol diacrylate, polyethylene glycol dimethacrylate, -N-vinylacrylamide, glycidyl acrylate, divinylbenzene, diallyldimethylammonium chloride, and tetraallyl ammonium chloride.

In various examples, photochemically-reversible hydrogel polymers and nanogel particles are prepared under free radical polymerization reaction conditions by reacting at least one first type of monomer, at least one second type of monomer, both in accordance with the above recited structures, and at least one multifunctional monomer. With these two types of monomers and a multifunctional monomer used in a free radical polymerization reaction, resulting photochemically-reversible hydrogel polymers and nanogel particles include copolymer chains having at least a first repeating unit incorporating the first type of monomer and at least a second repeating unit incorporating the second type of monomer, wherein the copolymer chains have at least some degree of crosslinking between copolymer chains. Additional crosslinking of copolymer chains, and/or bonding of photochemically-reversible hydrogel polymers or nanogel polymers to functionalized surfaces, may be achieved by photochemical dimerization of alkene or diene groups in the copolymer chains or between copolymer chains and functionalized surfaces.

In various examples, photochemically-reversible hydrogel polymers and nanogel particles are prepared under free radical polymerization reaction conditions by reacting at least one first type of monomer, at least one second type of monomer, both in accordance with the above recited structures, and the multifunctional monomer N,N′-methylenebismethacrylamide (BisAM).

In various examples, photochemically-reversible hydrogel polymers and nanogel particles thus prepared under free radical polymerization reaction conditions including at least one of each of two types of monomers and optionally a multifunctional monomer as described above include copolymer chains including at least a reactive alkene or 1,4-diene capable of [2+2] or [2+2+2+2] photodimerization, respectively, at wavelengths >270 nm, and at least one of a carboxylic acid, —N₃, or —C≡CH end group.

### Suspension/Precipitation and Other Free-Radical Polymerizations

In various examples, syntheses of photochemically-reversible nanogel particles include various aspects of suspension/precipitation free-radical polymerization or emulsion polymerization. In various embodiments, reaction conditions are aqueous and heated, employing selected monomers, a dispersant to facilitate suspension of generally water-insoluble photochemically-reversible nanogel particles thus formed in water, and a free-radical initiator.

In various examples, a suspension/precipitation free-radical polymerization reaction is conducted at a temperature of from about 50° C. to about 90° C., over the course of about 1 hour to 4 hours.

In various examples, a dispersant herein comprises an anionic or nonionic dispersant. Exemplary anionic dispersants included sodium dodecyl sulfate (SDS). Nonionic dispersants include, but are not limited to, polyethylene glycol (PEG), sorbitan monooleates (e.g., under the brand name Span®), ethoxylated sorbitan monooleates (e.g., under the brand name Tween®), and acryloyl-terminated PEG.

In various examples, a free-radical initiator includes a water-soluble compound.

In various examples, a free-radical initiator includes a peroxide.

In various examples, a free-radical initiator includes sodium, potassium, or ammonium persulfate.

In various examples, a free-radical initiator includes ammonium persulfate (APS).

In various examples, photochemically-reversible nanogel particles are synthesized in a suspension/precipitation free-radical polymerization reaction incorporating a dispersed monomer mixture including 2-((4-methyl-2-oxo-2H-chromen-7-yl)oxy)ethyl acrylate (CAA); N,N-dimethylacrylamide (NDMAM); and N-(5-(2-azidoacetamido)pentyl)acrylamide (AzAPA).

In various examples, photochemically-reversible nanogel particles are synthesized in a suspension/precipitation free-radical polymerization reaction incorporating a dispersed monomer mixture including 2-((4-methyl-2-oxo-2H-chromen-7-yl)oxy)ethyl acrylate (CAA); N,N-dimethylacrylamide (NDMAM); N-(5-(2-azidoacetamido)pentyl)acrylamide (AzAPA); and the multifunctional monomer N,N′-methylenebisacrylamide (BisAM).

In various examples, photochemically-reversible nanogel particles are synthesized in a suspension/precipitation free-radical polymerization reaction incorporating a dispersed monomer mixture including 2-((4-methyl-2-oxo-2H-chromen-7-yl)oxy)ethyl acrylate (CAA); N,N-dimethylacrylamide (NDMAM); N-(5-(2-azidoaetamido)pentyl)acrylamide (AzAPA); and acrylic acid (AAc).

In various examples, photochemically-reversible nanogel particles are synthesized in a suspension/precipitation free-radical polymerization reaction incorporating a dispersed monomer mixture including 2-((4-methyl-2-oxo-2H-chromen-7-yl)oxy)ethyl acrylate (CAA); N,N-dimethylacrylamide (NDMAM); N-(5-(2-azidoacetamido)pentyl)acrylamide (AzAPA); acrylic acid (AAc); and the multifunctional monomer N,N′-methylenebisacrylamide (BisAM).

In various examples, photochemically-reversible nanogel particles are synthesized in a suspension/precipitation free-radical polymerization reaction incorporating a dispersed monomer mixture including 2-((4-methyl-2-oxo-2H-chromen-7-yl)oxy)ethyl acrylate (CAA), N-isopropylacrylamide (NiPAM); N-(5-(2-azidoacetamido)pentyl)acrylamide (AzAPA); and acrylic acid (AAc).

In various examples, photochemically-reversible nanogel particles are synthesized in a suspension/precipitation free-radical polymerization reaction incorporating a dispersed monomer mixture including 2-((4-methyl-2-oxo-2H-chromen-7-yl)oxy)ethyl acrylate (CAA), N-isopropylacrylamide (NiPAM); N-(5-(2-azidoacetamido)pentyl)acrylamide (AzAPA); acrylic acid (AAc); and the multifunctional monomer N,N′-methylenebisacrylamide (BisAM).

In various examples, photochemically-reversible nanogel particles are synthesized in a suspension/precipitation free-radical polymerization reaction incorporating a dispersed monomer mixture including N-(5-(2-azidoacetamido)pentyl)acrylamide (AzAPA) and acrylic acid (AAc) to form copolymer chains, followed by reaction of at least some of the available —N₃ end groups on the resulting copolymer chains with N-(but-3-yn-1-yl)-2-(((2-oxo-2H-chromen-7-yl)oxy)methyl)acrylamide (alkyne coumarin).

In various examples, photochemically-reversible nanogel particles are synthesized in a suspension/precipitation free-radical polymerization reaction incorporating a dispersed monomer mixture including N-(5-(2-azidoacetamido)pentyl)acrylamide (AzAPA), acrylic acid (AAc), and the multifunctional monomer N,N′-methylenebisacrylamide (BisAM) to form copolymer chains, followed by reaction of at least some of the available —N₃ end groups on the resulting copolymer chains with N-(but-3-yn-1-yl)-2-(((2-oxo-2H-chromen-7-yl)oxy)methyl)acrylamide (alkyne coumarin).

In various examples, photochemically-reversible nanogel particles are synthesized in a suspension/precipitation free-radical polymerization reaction incorporating a dispersed monomer mixture including N,N-dimethylacrylamide (NDMAM), N-(5-(2-azidoacetamido)pentyl)acrylamide (AzAPA) and acrylic acid (AAc) to form copolymer chains, followed by reaction of at least some of the available —N₃ end groups on the resulting copolymer chains with N-(but-3-yn-1-yl)-2-(((2-oxo-2H-chromen-7-yl)oxy)methyl)acrylamide (alkyne coumarin).

In various examples, photochemically-reversible nanogel particles are synthesized in a suspension/precipitation free-radical polymerization reaction incorporating a dispersed monomer mixture including N,N-dimethylacrylamide (NDMAM), N-(5-(2-azidoacetamido)pentyl)acrylamide (AzAPA), acrylic acid (AAc), and the multifunctional monomer N,N′-methylenebisacrylamide (BisAM) to form copolymer chains, followed by reaction of at least some of the available —N₃ end groups on the resulting copolymer chains with N-(but-3-yn-1-yl)-2-(((2-oxo-2H-chromen-7-yl)oxy)methyl)acrylamide (alkyne coumarin).

In various examples, photochemically-reversible nanogel particles are synthesized in a suspension/precipitation free-radical polymerization reaction incorporating a dispersed monomer mixture including N-isopropylacrylamide (NiPAM), N-(5-(2-azidoacetamido)pentyl)acrylamide (AzAPA) and acrylic acid (AAc) to form copolymer chains, followed by reaction of at least some of the available —N₃ end groups on the resulting copolymer chains with N-(but-3-yn-1-yl)-2-(((2-oxo-2H-chromen-7-yl)oxy)methyl)acrylamide (alkyne coumarin).

In various examples, photochemically-reversible nanogel particles are synthesized in a suspension/precipitation free-radical polymerization reaction incorporating a dispersed monomer mixture including N-isopropylacrylamide (NiPAM), N-(5-(2-azidoacetamido)pentyl)acrylamide (AzAPA), acrylic acid (AAc), and the multifunctional monomer N,N′-methylenebisacrylamide (BisAM) to form copolymer chains, followed by reaction of at least some of the available —N₃ end groups on the resulting copolymer chains with N-(but-3-yn-1-yl)-2-(((2-oxo-2H-chromen-7-yl)oxy)methyl)acrylamide (alkyne coumarin).

### Photochemically-Reversible Hydrogel Polymers and Nanogel Particles Including Copolymer Chains

In various examples, photochemically-reversible hydrogel polymers and nanogel particles include copolymer chains having various end groups on at least some of the copolymer chains. In various examples, photochemically-reversible hydrogel polymers and nanogel particles include copolymer chains having at least some degree of crosslinking. In various examples, at least some degree of crosslinking is obtained photochemically by dimerizing certain reactive alkene or 1,4-diene end groups on copolymer chains.

In various examples, photochemically-reversible hydrogel polymers and polymeric nanogel particles in accordance with the present disclosure include copolymer chains further including a first recurring unit of Formula (I):

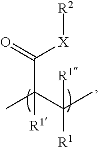

wherein:
each of R¹, R^{1′}, and R^{1″} is independently selected from H, halogen, alkyl, alkoxy, alkenyl, alkynyl, cycloalkyl, aryl, heteroaryl, or heterocyclyl;
X is —O— or —NH—; and
R² is —CH₂—C≡CH, or R² has a structure:

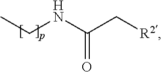

wherein R^{2′} is —NH₂, alkyl, alkoxy, alkenyl, alkynyl, or optionally substituted variants thereof, or halogen, —N₃, —OH, —C(O)H, —NH═NH₂, —SCN, —CO₂H, —SH, glycidyl, epoxy, aziridine, triazoline, or —C≡CH; and
p is an integer of 1 to 50; and
a second recurring unit of Formula (II),

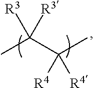

wherein:
each of R³, R^{3′}, R⁴, and R^{4′} is independently selected from —H, —R⁵, —OR⁵, —CO₂R⁵, —C(O)R⁵, —OC(O)R⁵, —C(O)NR⁶R⁷, —NR⁶R⁷ or a substructure of Formula (III),

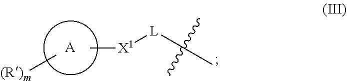

R⁵ is —H, —OH, alkyl, cycloalkyl, hydroxyalkyl, aryl, heteroaryl, or heterocyclyl;
each of R⁶ and R⁷ is independently selected from —H and alkyl;
A is an aryl or a thymidinyl moiety;
R′ is —H, alkyl, alkoxy, alkenyl, alkynyl, aryl, heterocyclyl or optionally substituted variants thereof, or halogen, —N₃, —OH, —C(O)H, —NH═NH₂, —SCN, —CO₂H, —SH, glycidyl, epoxy, or two R′ groups, when bonded to adjacent atoms on ring A and taken together with ring A, form a coumarinyl, an anthracenyl, an acenaphthylenyl, a thianaphthenyl-1-oxide or a thianaphthenyl -1,1-dioxide moiety;
X¹ is a bond, —(CH₂)_{q}—, —O— or —NH—;
L is a bivalent linker having a structure —(CH₂)_{q}—X²—C(═O)— or —(CH₂CH₂O)_{q}—X²—C(═O)—;
X² is —O— or —NH—;
m is an integer from 1 to 9; and
q is an integer from 0 to 50.

In various examples, R¹═R^{1′}═R^{1″}═H; X is —O— or —NH—; R² is —CH₂—C≡CH or has a structure:

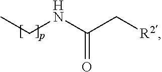

wherein R^{2′} is —N₃ or —C≡CH; and p is an integer of 1 to 50.

In various examples of the second repeating unit of Formula (II), Formula (III) has the subgenus structure of Formula (IV):

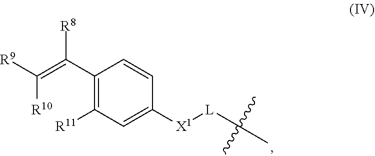

wherein each of R⁸ and R⁹ is —H, alkyl, cycloalkyl, hydroxyalkyl, aryl, heteroaryl, or heterocyclyl; each R¹⁰ and R¹¹ is independently -H, alkyl, cycloalkyl, hydroxyalkyl, aryl, heteroaryl, or heterocyclyl, or R¹⁰ is —C(═O)—, R¹¹ is —O—, and R¹⁰ and R¹¹ are bonded together such that Formula (IV) comprises a substituted coumarin moiety.

In various examples, photochemically-reversible hydrogel polymers and nanogel particles having the above-recited recurring units include copolymer chains having at least one reactive alkene or reactive 1,4-diene end group capable of [2+2] or [2+2+2+2] photodimerization, respectively, at wavelengths >270 nm.

In various examples, photochemically-reversible hydrogel polymers and nanogel particles having the above-recited recurring units include copolymer chains having (1) at least one reactive alkene or reactive 1,4-diene end group capable of [2+2] or [2+2+2+2] photodimerization, respectively, at wavelengths >270 nm; and (2) at least one of a carboxylic acid, —N₃, or —C≡CH end group.

In various examples, photochemically-reversible hydrogel polymers and nanogel particles having the above-recited recurring units include copolymer chains having at least one carboxylic acid end group, at least one —N₃ end group, and at least one reactive alkene or reactive 1,4-diene end group capable of [2+2] or [2+2+2+2] photodimerization, respectively, at wavelengths >270 nm.

In various examples, photochemically-reversible hydrogel polymers and nanogel particles include copolymer chains wherein the first recurring unit of Formula (I) is:

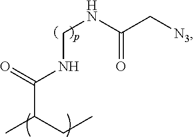

wherein p, as per above, is an integer of 1 to 50.

In various examples, photochemically-reversible hydrogel polymers and nanogel particles include copolymer chains wherein the first recurring unit of Formula (I) is:

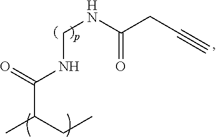

wherein p, as per above, is an integer of 1 to 50.

In various examples, photochemically-reversible hydrogel polymers and nanogel particles include copolymer chains wherein the first recurring unit of Formula (I) is:

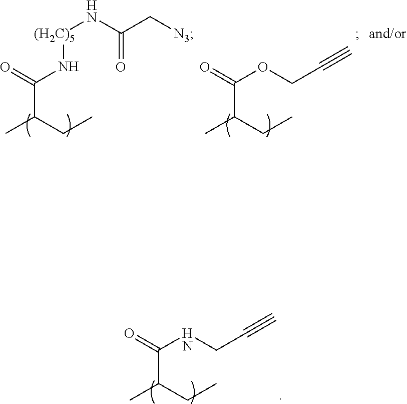

In various examples, photochemically-reversible hydrogel polymers and nanogel particles include copolymer chains wherein the second recurring unit of Formula (II) is:

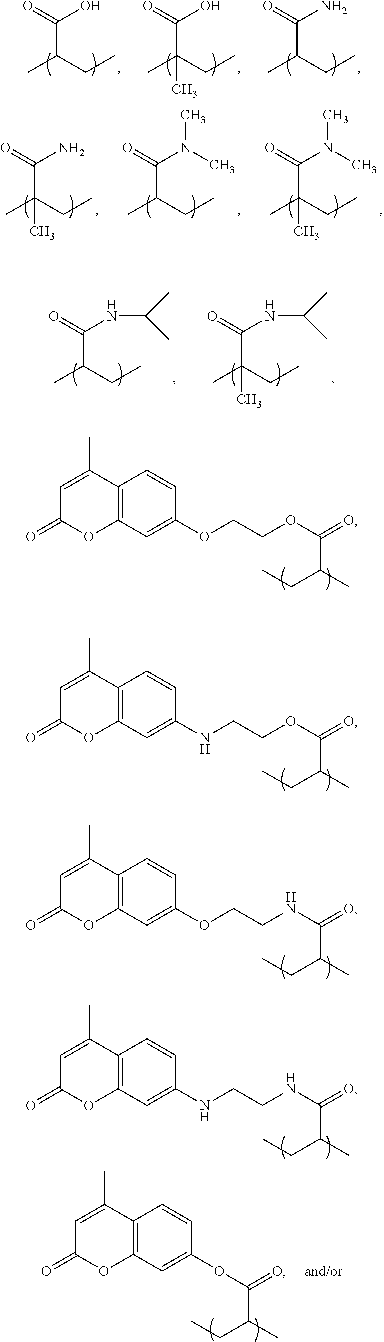

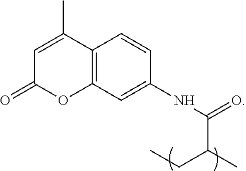

In various examples, photochemically-reversible hydrogel polymers and nanogel particles include copolymer chains wherein the second recurring unit of Formula (II) is:

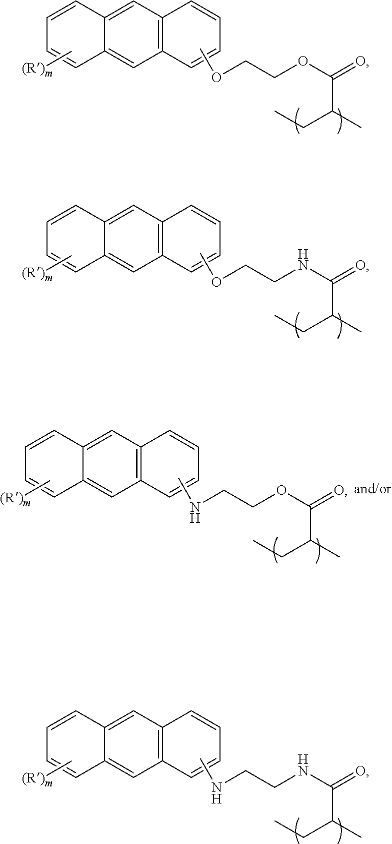

wherein R′ is —H, alkyl, alkoxy, alkenyl, alkynyl, aryl, heterocyclyl or optionally substituted variants thereof, or halogen, —N₃, —OH, —C(O)H, —NH═NH₂, —SCN, —CO₂H, —SH, glycidyl, epoxy; and,
m is an integer from 1 to 9.

In various examples, photochemically-reversible hydrogel polymers and nanogel particles include copolymer chains wherein the second recurring unit of Formula (II) is:

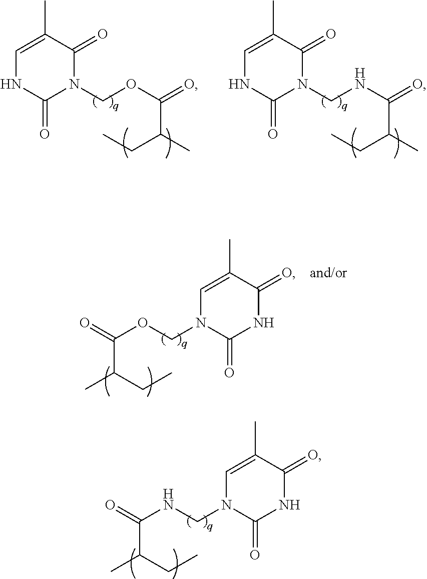

wherein q is an integer from 0 to 50.

In various examples, photochemically-reversible hydrogel polymers and nanogel particles include copolymer chains wherein the second recurring unit of Formula (II) is:

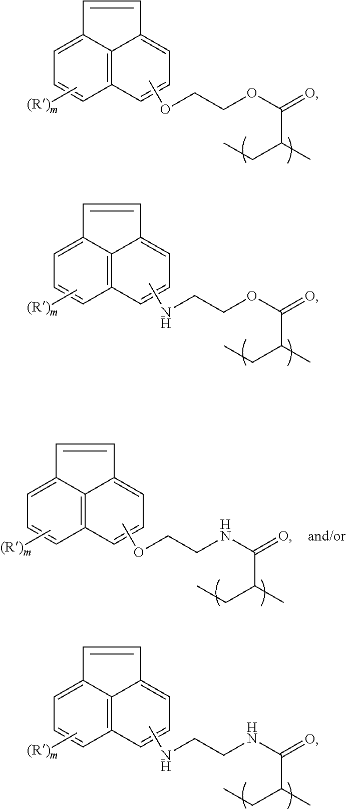

wherein R′ is —H, alkyl, alkoxy, alkenyl, alkynyl, aryl, heterocyclyl or optionally substituted variants thereof, or halogen, —N₃, —OH, —C(O)H, —NH═NH₂, —SCN, —CO₂H, —SH, glycidyl, epoxy; and,
m is an integer from 1 to 7.

In various examples, photochemically-reversible hydrogel polymers and nanogel particles include copolymer chains wherein the second recurring unit of Formula (II) is:

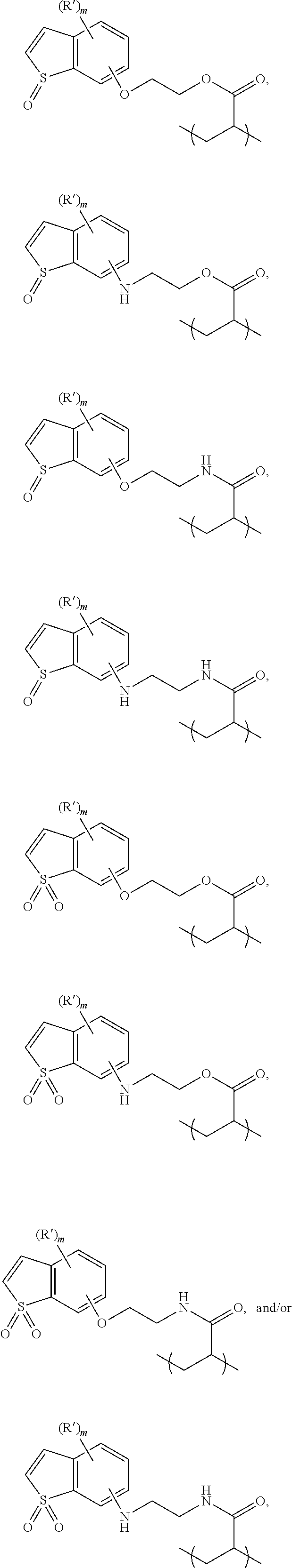

wherein R′ is —H, alkyl, alkoxy, alkenyl, alkynyl, aryl, heterocyclyl or optionally substituted variants thereof, or halogen, —N₃, —OH, —C(O)H, —NH═NH₂, —SCN, —CO₂H, —SH, glycidyl, epoxy; and,
m is an integer from 1 to 5.

In various examples, photochemically-reversible hydrogel polymers and nanogel particles include copolymer chains wherein the second recurring unit of Formula (II) is:

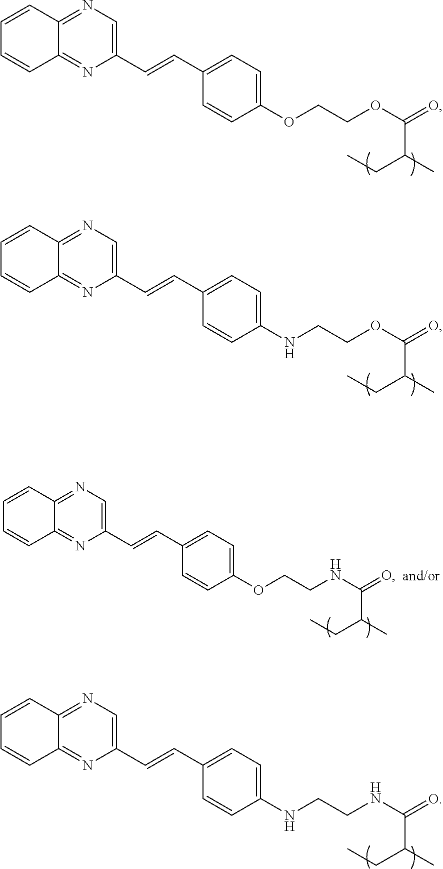

In various examples, photochemically-reversible hydrogel polymers and nanogel particles include poly(NiPAM-co-AzAPA-co-AAc) copolymer chains functionalized with alkyne coumarin such that at least some of these copolymer chains include unused —N₃ end groups, unreacted —CO₂H end groups, and at least some N-(2-(1λ²2,2,3-triazol-4-yl)ethyl)-2-4(2-oxo-2H -chromen-7-yl)oxy)methyl)acrylamide appendages providing reactive alkene end groups capable of [2+2] photodimerization at wavelengths >270 nm.

In various examples, photochemically-reversible hydrogel polymers and nanogel particles include poly(NiPAM-co-AzAPA-co-AAc-co-BisAM) copolymer chains functionalized with alkyne coumarin such that at least some of these copolymer chains include unused —N₃ end groups, unreacted —CO₂H end groups, and at least some N-(2-(1λ²,2,3-triazol-4-yl)ethyl)-2-(((2-oxo-2H-chromen-7-yl)oxy)methyl)acrylamide appendages providing reactive alkene end groups capable of [2+2] photodimerization at wavelengths >270 nm.

In various examples, photochemically-reversible hydrogel polymers and nanogel particles include poly(NDMAM-co-AzAPA-co-AAc) copolymer chains functionalized with alkyne coumarin such that at least some of these copolymer chains include unused —N₃ end groups, unreacted —CO₂H end groups, and at least some N-(2-(1λ²,2,3-triazol-4-yl)ethyl)-2-4(2-oxo-2H -chromen-7-yl)oxy)methyl)acrylamide appendages providing reactive alkene end groups capable of [2+2] photodimerization at wavelengths >270 nm.

In various examples, photochemically-reversible hydrogel polymers and nanogel particles include poly(NDMAM-co-AzAPA-co-AAc-co-BisAM) copolymer chains functionalized with alkyne coumarin such that at least some of these copolymer chains include unused —N₃ end groups, unreacted —CO₂H end groups, and at least some N-(2-(1λ²,2,3-triazol-4-yl)ethyl)-2-(((2-oxo-2H-chromen-7-yl)oxy)methyl)acrylamide appendages providing reactive alkene end groups capable of [2+2] photodimerization at wavelengths >270 nm.

In various examples, photochemically-reversible hydrogel polymers and nanogel particles include poly(CAA-co-NDMAM-co-AAc) copolymer chains. In various examples, at least some of these copolymer chains include —CO₂H end groups and reactive alkene end groups capable of [2+2] photodimerization at wavelengths >270 nm for dual functionality.

In various examples, photochemically-reversible hydrogel polymers and nanogel particles include poly(CAA-co-NDMAM-co-AAc-co-BisAM) copolymer chains. In various examples, at least some of these copolymer chains include —CO₂H end groups and reactive alkene end groups capable of [2+2] photodimerization at wavelengths >270 nm for dual functionality.

In various examples, photochemically-reversible hydrogel polymers and nanogel particles include poly(CAA-co-NDMAM-co-AzAPA) copolymer chains. In various examples, at least some of these copolymer chains include —N₃ end groups and reactive alkene end groups capable of [2+2] photodimerization at wavelengths >270 nm for dual functionality.

In various examples, photochemically-reversible hydrogel polymers and nanogel particles include poly(CAA-co-NDMAM-co-AzAPA-co-BisAM) copolymer chains. In various examples, at least some of these copolymer chains include —N₃ end groups and reactive alkene end groups capable of [2+2] photodimerization at wavelengths >270 nm for dual functionality.

In various examples, photochemically-reversible hydrogel polymers and nanogel particles include poly(CAA-co-NDMAM-co-AzAPA-co-AAc) copolymer chains. In various examples, at least some of these copolymer chains include —N₃ end groups, —CO₂H end groups, and reactive alkene end groups capable of [2+2] photodimerization at wavelengths >270 nm for dual functionality.

In various examples, photochemically-reversible hydrogel polymers and nanogel particles include poly(CAA-co-NDMAM-co-AzAPA-co-AAc-co-BisAM) copolymer chains. In various examples, at least some of these copolymer chains include —N₃ end groups, —CO₂H end groups, and reactive alkene end groups capable of [2+2] photodimerization at wavelengths >270 nm for dual functionality.

In various examples, photochemically-reversible hydrogel polymers and nanogel particles include poly(CAA-co-NiPAM-co-AzAPA-co-AAc) copolymer chains. In various examples, at least some of these copolymer chains include —N₃ end groups, —CO₂H end groups, and reactive alkene end groups capable of [2+2] photodimerization at wavelengths >270 nm for dual functionality.

In various examples, photochemically-reversible hydrogel polymers and nanogel particles include poly(CAA-co-NiPAM-co-AzAPA-co-AAc-co-BisAM) copolymer chains. In various examples, at least some of these copolymer chains include —N₃ end groups, —CO₂H end groups, and reactive alkene end groups capable of [2+2] photodimerization at wavelengths >270 nm for dual functionality.

In various examples, photochemically-reversible hydrogel polymers and nanogel particles include poly(PAG-co-CAA-co-NiPAM-co-AAc) copolymer chains. In various examples, at least some of these copolymer chains include —C≡CH end groups, —CO₂H end groups, and reactive alkene end groups capable of [2+2] photodimerization at wavelengths >270 nm for dual functionality.

In various examples, photochemically-reversible hydrogel polymers and nanogel particles include poly(PAG-co-CAA-co-NiPAM-co-AAc-co-BisAM) copolymer chains. In various examples, at least some of these copolymer chains include —C≡CH end groups, —CO₂H end groups, and reactive alkene end groups capable of [2+2] photodimerization at wavelengths >270 nm for dual functionality.

### Grafting Amplification Primers Onto Photochemically-Reversible Hydrogel Polymers and Nanogel Particles

The above general examples show that photochemically-reversible hydrogel polymers and nanogel particles can be prepared wherein copolymer chains of the photochemically-reversible hydrogel polymers and nanogel particles include at least some reactive alkene or 1,4-diene end groups capable of [2+2] or [2+2+2+2] photodimerization, respectively, at wavelengths >270 nm, and at least some —C≡CH end groups, —CO₂H end groups, and/or —N₃ end groups. In some examples, the —CO₂H end groups of the copolymer chains are leveraged in attaching photochemically-reversible hydrogel polymers or nanogel particles to surfaces such as lanes within a FC used for SBS. Further, the —N₃ end groups or —C≡CH end groups of the copolymer chains may be leveraged in grafting suitably functionalized amplification primers (such as P5/P7) onto photochemically-reversible hydrogel polymers or each nanogel particle in preparation for SBS. In some examples, [2+2] or [2+2+2+2] photodimerization is used to attach photochemically-reversible hydrogel polymers or nanogel particles to a suitably functionalized FC surface in preparation for SBS.

In various examples, grafting of an amplification primer to photochemically-reversible hydrogel polymers or nanogel particles includes click-chemistry between a terminal alkyne substituent on the amplification primer and an —N₃ end group of a respective copolymer chain, or click-chemistry between a terminal —N₃ substituent on the amplification primer and a —C≡CH end group on a respective copolymer chain. Stated another way, grafting of amplification primers to photochemically-reversible hydrogel polymers or nanogel particles in various examples includes alkyne-azide or azide-alkyne cycloaddition click chemistry, covalently linking primer to nanogel particle through a triazine moiety.

In other aspects, thiol-functionalized primers may be grafted onto photochemically-reversible hydrogel polymers or nanogel particles including copolymer chains wherein at least some of the copolymer chains include —C≡CH end groups.

In various examples, and as detailed above, the choice of monomers used in the synthesis of the photochemically-reversible hydrogel polymers or nanogel particles dictates whether the resulting copolymer chains include —N₃ or —C≡CH end groups in addition to alkene or 1,4-diene and optional —CO₂H end groups. For grafting purposes, a complementary functional group is chosen for the functionalized amplification primer to promote click-chemistry.

P5 and P7 amplification primers for use herein are used on the surface of commercial flow cells sold by Illumina Inc. for sequencing on the HiSeq™, MiSeq™, NextSeq™ and Genome Analyzer™ platforms. P5/P7 amplification primers for grafting are fully described in U.S. Pat. No. 9,982,250 and U.S. Publication No. 2011/0059865, the disclosures of which are incorporated herein by reference in their entireties.

In various examples, functionalized amplification primers for grafting onto photochemically-reversible hydrogel polymers or nanogel particles include, but are not limited to, alkyne-P5/P7 primers, N₃-P5/P7 primers, and thiol-P5/P7 primers.

In various examples, grafting of alkyne-P5/P7 primers onto photochemically-reversible hydrogel polymers or nanogel particles that include copolymer chains having —N₃ end groups includes CuAAC grafting, resulting in P5/P7-grafted photochemically-reversible hydrogel polymers or nanogel particles.

In various examples, CUAAC catalyzed click chemistry involving N₃-P5/P7 or thiol-P5/P7 primers is conducted at a temperature of from about 40° C. to about 80° C., and from about 1 hour to about 5 hours.

In various examples, grafting of N₃-P5/P7 or thiol-P5/P7 primers onto photochemically-reversible hydrogel polymers or nanogel particles that include copolymer chains having —C≡CH end groups includes CuAAC grafting, resulting in P5/P7-grafted photochemically-reversible hydrogel polymers or nanogel particles.

### Capture of Primer-Grafted Photochemically-Reversible Nanogel Particles for SBS

In various examples, and as part of an SBS method, primer-grafted photochemically-reversible nanogel particles are captured on surfaces in a flow cell (FC) such as the HiSeq™ FC from Illumina, Inc. Primer-grafted photochemically-reversible nanogel particles may be captured into nano-wells patterned in coatings on the FC surface, or directly attached to coatings on surfaces absent nano-wells. In various examples, each primer-grafted nanogel particle can act as a nano-well and thus can function as a replacement for the same.

In various examples, primer-grafted photochemically-reversible nanogel particles are captured into nano-wells of a FC by either:
(a) a bioconjugation technique using DMTMM to activate reaction between the free carboxylate end groups present on the copolymer chains of the primer-grafted photochemically-reversible nanogel particles and —NH₂ groups available on a previously silanized FC surface to form amide linkages, or
(b) CuAAC click-chemistry to react any remaining —C≡CH end groups still present on copolymer chains of the primer-grafted photochemically-reversible nanogel particles (i.e., after grafting) with reactive —N₃ groups present on a standard PAZAM coated and polished FC to form triazine linkages.

In various examples relating to (a) above, silanization of a FC surface may be accomplished using any suitable silane or silane derivative. The method used to attach a silane or silane derivative to a substrate may vary depending upon the silane or silane derivative that is being used.

In various examples, the silane or silane derivative is 3-mercaptopropylsilanetriol, 3-aminopropyltriethoxysilane (APTES) or 3-aminopropyltrimethoxysilane (APTMS) (i.e., silanes having the general structure, X—R^{B}—Si(OR^{C})₃, wherein X is amino, R^{B} is —(CH₂)₃—, and R^{C} is H, ethyl or methyl). In this example, a FC surface may be pre-treated with APTES or APTMS to covalently link silicon to one or more oxygen atoms on the surface. This chemically treated surface is optionally baked to form an amine group monolayer.

In various examples, the plurality of —NH₂ groups present on a FC surface are then reacted with carboxylate end groups present on corresponding copolymer chains of the primer-grafted photochemically-reversible nanogel particles. This procedure takes advantage of the dual functionality of the nanogel particles in that the —CO₂H end groups are only used to bind the nanogel particles to the FC surfaces while either —N₃ or —C≡CH end groups were previously used only for grafting functionalized primers to the nanogel particles.

In various examples relating to (b) above, PAZAM coatings on FC surfaces are prepared using N-(5-(2-bromoacedamido)pentyl)acrylamide (BrAPA) as a monomer for polymeric hydrogel coating, followed by conversion of the bromo groups to —N₃ groups.

In various examples, PAZAM may be deposited on the surface of a patterned FC surface by spin coating, dipping, dip coating, or flow of the PAZAM under positive or negative pressure, or another suitable technique. The PAZAM may be present in a mixture. In an example, the mixture includes PAZAM in water or in an ethanol and water mixture.

After being coated, the functionalized molecule may also be exposed to a curing process to form the functionalized coating layer across the entire patterned substrate (i.e., on depression(s) and interstitial region(s)). In an example, curing the functionalized molecule may take place at a temperature ranging from room temperature (e.g., about 25° C.) to about 60° C. for a time ranging from about 5 minutes to about 2 hours.

To form a PAZAM coating layer in nano-wells and not on interstitial regions of a patterned substrate, the PAZAM coating layer may be polished off of the interstitial regions using either (a) a basic, aqueous slurry having a pH ranging from about 7.5 to about 11 and including an abrasive particle, or (b) a polishing pad and a solution free of an abrasive particle.

To capture photochemically-reversible nanogel particles onto PAZAM coated FC surfaces, the PAZAM coating having reactive —N₃ groups can react with any remaining —C≡CH end groups present on copolymer chains of the photochemically-reversible nanogel particles under conditions for CuAAC click-chemistry. For any additional details, see the '033 Patent (Illumina) referenced above and incorporated herein.

In alternative examples, the order of the distinct steps of primer grafting and particle capture can be reversed. Thus, photochemically-reversible nanogel particles having dual functionality may be captured on silanized FC surfaces or PAZAM coated FC surfaces by amide formation or click-chemistry, and then the captured particles are subsequently exposed to suitably functionalized amplification primers (alkyne-P5/P7 or N₃-P5/P7, for example) to append the amplification primers to the captured nanogel particles.

In alternative examples, photochemically-reversible nanogel particles may be reversibly attached to suitably functionalized FC surfaces by either [2+2] or [2+2+2+2] photodimerization. For example, a FC surface may be silanized with 3-mercaptopropylsilanetriol, 3-mercaptopropyltrimethoxysilane or 3-mercaptopropyltriethoxysilane to tether a plurality of —SH groups to the FC surface. Then, compounds having both acrylate functionality and either a reactive alkene or reactive 1,4-diene moiety (e.g., N-(2-((4-methyl-2-oxo-2H-chromen-7-yl)oxy)ethyl)acrylamide) are reacted with the tethered —SH groups in thiol-ene (1,4-addition) reactions, thus converting the tethered —SH functionality to a plurality of tethered reactive alkene or 1,4-diene groups. Subsequently, these tethered reactive alkene or 1,4-diene groups are available for [2+2] or [2+2+2+2] photodimerization with photochemically-reversible nanogel particles having copolymer chains with reactive alkene or 1,4-diene end groups, respectively, preferably upon exposure to >270 nm incident radiation. An important feature of this method of polymer attachment is that it is reversible, such as upon exposure to incident radiation of <300 nm wavelength.

### Seeding, Clustering and SBS Sequencing

In various examples, clustering includes either suspension clustering or on-board clustering. On-board clustering may be used for proof-of-concept since suspension clustering avoids the need for patterning coated FC surfaces and each photochemically-reversible nanogel particle captured on the FC surface functions as its own nano-well. In suspension clustering, seeded ssDNA may be clustered on the surfaces of the photochemically-reversible nanogel particles. Clustering on photochemically-reversible nanogel particles is dependent on having sufficiently accessible primers grafted onto the photochemically-reversible nanogel particles.

In various examples, the temperature responsiveness of primer-grafted photochemically-reversible nanogel particles having blocks of poly(NiPAM) within copolymer chains, allows for temperature-controlled manipulation of seeding, amplification, and sequencing by:
(a) promoting temperature-controlled shrinkage during seeding to reduce the probability for multiple seeding events and therefore enhance monoclonality;
(b) promoting temperature-controlled swelling during clustering to increase primer accessibility and facilitate diffusion of materials into photochemically-reversible nanogel particles, resulting in an increased number of strands per cluster/particles and improvement its fluorescent; and/or
(c) promoting temperature-controlled shrinking or swelling to improve the SBS steps of incorporation and cleavage.

In various examples, FCs having captured primer-grafted photochemically-reversible nanogel particles are then used in a variety of sequencing approaches or technologies, including SBS, cyclic-array sequencing, sequencing-by-ligation, pyrosequencing, and so forth. With any of these techniques, since the sequencing primers are present only on the photochemically-reversible nanogel particles, amplification will be confined to each particle. Moreover, due to confinement of amplification to particle surfaces, there is more time to amplify one sequencing template into larger clusters.

In various examples, SBS may be run on a system such as the HISEQ™, HISEQX™, MISEQ™, NOVASEQ™, or NEXTSEQ™ sequencer systems (Illumina, Inc.). In SBS, extension of a nucleic acid primer along a nucleic acid template (i.e., the sequencing template) is monitored to determine the sequence of nucleotides in the template. The underlying chemical process can be polymerization (e.g., catalyzed by a polymerase enzyme) or ligation (e.g., catalyzed by a ligase enzyme). In various polymerase based SBS processes, fluorescently labeled nucleotides are added to the primer to extend the primer in a template dependent fashion such that detection of the order and type of nucleotides added to the primer can be used to determine the sequence of the template. For example, to initiate a first SBS cycle, one or more labeled nucleotides, DNA polymerase, etc., may be delivered into/through a flow channel in the FC that houses an array of primers on nanogel particles. The primer-grafted nanogel particles whereupon primer extension causes a labeled nucleotide to be incorporated, can be detected through an imaging event. During an imaging event, an illumination system provides an excitation light to the nanogel particles.

In various examples, nucleotides can further include a reversible termination property that terminates further primer extension once a nucleotide has been added to the primer. For example, a nucleotide analog having a reversible terminator moiety can be added to the primer such that subsequent extension cannot occur until a deblocking agent is delivered to remove the moiety. Thus, for examples that use reversible termination, a deblocking reagent can be delivered to a flow channel before or after detection.

Flushes (i.e., washes) may take place between the various fluid delivery steps. The SBS cycle can then be repeated n times to extend the primer by n nucleotides, thereby detecting a sequence of length n.

### Attachment of Photochemically-Reversible Hydrogel Polymers for SBS

In various examples, two distinct methods may be used to form photochemically-reversible hydrogel layers on surfaces of FCs to be used for SBS sequencing:
(1) provide a monomer and crosslinker compound mixture to a FC surface and irradiate the monomer and crosslinker compound mixture with wavelength >270 nm to form the hydrogel, crosslink at least some of the copolymer chains and effectively cure the hydrogel to the FC surface; or,
(2) provide a photochemically-reversible hydrogel polymer in accordance to the present disclosure to a suitably functionalized FC and irradiate the FC surface to bond the hydrogel polymer to the functionalized FC surface through [2+2] or [2+2+2+2] photodimerization.

For method (1), termed “in-FC crosslinking”), the monomer mixture is in accordance with the present disclosure and is therefore a mixture of at least a first type of monomer and at least a second type of monomer as set forth herein. A crosslinker compound added into the monomer mixture may be a multifunctional monomer such as N,N′-methylenebisacrylamide, N,N′-methylenebismethacrylamide, polyethylene glycol diacrylate, polyethylene glycol dimethacrylate, N-vinylacrylamide, glycidyl acrylate, divinyibenzene, or tetraallyl ammonium chloride, or other known substances used for crosslinking.

For method (2) the FC surface is functionalized by silanization with 3-mercaptopropylsilanetriol, 3-mercaptopropyltrimethoxysilane or 3-mercaptopropyltriethoxysilane to tether a plurality of —SH groups to the FC surface. Then, compounds having both acrylate functionality and either a reactive alkene or reactive 1,4-diene moiety (e.g., N-(2-((4-methyl-2-oxo-2H-chromen-7-yl)oxy)ethyl)acrylamide) are reacted with the tethered —SH groups in thiol-ene (1,4-addition) reactions, thus converting the tethered —SH functionality to a plurality of tethered reactive alkene or 1,4-diene groups. Subsequently, these tethered reactive alkene or 1,4-diene groups are available for [2+2] or [2+2+2+2] photodimerization with photochemically-reversible hydrogels having copolymer chains with reactive alkene or 1,4-diene end groups, respectively. Preferably the incident radiation used to attach the hydrogel to the functionalized surface has wavelength >270 nm.

### Detachment of Photochemically-Reversible Hydrogel Polymers and Nanogel Particles to Reuse FCs Previously Used for SBS

In various examples, both photochemically-reversible hydrogels and photochemically-reversible nanogel particles are removable from FC surfaces. Previously, removal of hydrogels or nanogel particles from FC surfaces required use of harsh chemicals. In accordance with the present disclosure, photochemically-reversible hydrogels and photochemically-reversible nanogel particles are easily removed from FC surfaces without the use of harsh chemicals, simply by irradiation of the surfaces with radiation having wavelength <300 nm. The irradiation cleaves the [2+2] or [2+2+2+2] dimers, releasing the photochemically-reversible hydrogel or photochemically-reversible nanogel particles from the FC surfaces. Further cleaning of the FC surface, such as to remove the tethered silane groups, requires only mild conditions, such as mild acid rinsing.

In various examples, a sequencing/reuse workflow can be described as follows:
(1) The FC surface can be functionalized with a photo-crosslinking motif.
(2) Photochemically reversible hydrogels or nanogel particles containing a complementary photo-crosslinking motif can be flushed through the FC. The hydrogel or nanogel particles comprise amplification primers grafted thereon. Alternatively, in-suspension clustered nanogel particles containing photo-crosslinking motifs can also be used.
(3) The FC is subjected to irradiation of wavelength λ₁ which induces photo-crosslinking of the hydrogel or the nanogel particles to the FC surface.
(4) Once captured on the FC surface, clustering and sequencing are performed.
(5) Upon completion of all nucleic acid sequencing steps, the FC is subjected to a second irradiation comprising a second wavelength λ₂. This wavelength induces the photo-cleavage of the crosslinked motifs.
(6) Upon completion of the photo-cleavage, the FC is flushed through with a wash solution to remove sequenced materials.
(7) The FC can then be re-used.

### Long Wavelength Photochemically-Switchable Chemistries

In various examples, photochemically-reversible hydrogel polymers and polymeric nanogel particles may employ long wavelength (650 to 1100 nm) photochemically-switchable chemistry rather than short wavelength photochemically-switchable chemistry.

In various examples, photochemically-reversible hydrogel polymers and polymeric nanogel particles may comprise copolymer chains having azobenzene moieties capable of photoisomerization between E and Z isomers in accordance with the following scheme:

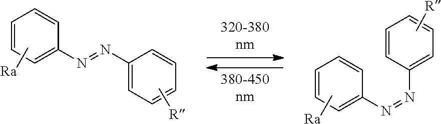

In various examples, the photoisomerization between E and Z isomers can be used to shift copolymer chains off surfaces, such as through electrostatic changes effecting bonding of monolayers. In various embodiments, R″ substituents on the azobenzene moieties may include oleyl amides or esters, wherein the oleyl chain provides van der Waals interactions that support self-assembled monolayers (SAMs). The substituent Ra may be the remaining portion of the copolymer chain of the hydrogel polymer or polymeric nanogel particle. In various examples, Ra and R″ may be connected in the same or in different polymer chains.

In various examples, photochemically-reversible hydrogel polymers and polymeric nanogel particles may comprise copolymer chains having spiropyran moieties capable of photoisomerization between open merocyanine and closed spiropyran isomers in accordance with the following scheme:

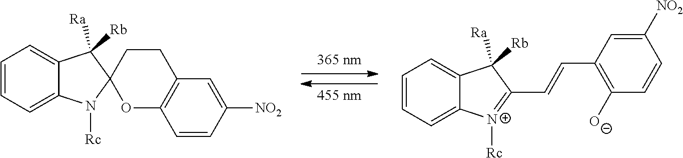

In various examples, the photochromism in the above scheme can be used to attach/detach hydrogel polymers or polymeric nanogel particles to/from surfaces, such as by altering electrostatic interactions between polymer and surface. In various examples, at least one of Ra, Rb or Rc may be the remaining portion of the copolymer chain of the hydrogel polymer or polymeric nanogel particles. In alternative embodiments, one portion of the merocyanine may be in one set of copolymer chains and another portion in a second set of copolymer chains such that the photochromism to cyclize the merocyanine connects copolymer chains together or connects copolymer chains to functionalized surfaces of a substrate.

In various examples, photochemically-reversible hydrogel polymers and polymeric nanogel particles may comprise copolymer chains having conjugated bis-thiophene ethylene moieties capable of reversible 2+2+2 cycloaddition in accordance with the following scheme:

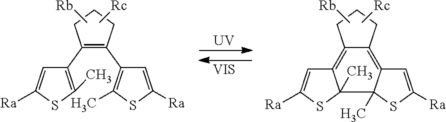

In various examples, the reversible photocycloaddition in the above scheme can be used to attach/detach hydrogel polymers or polymeric nanogel particles to/from surfaces, such as by altering electrostatic interactions between polymer and surface. In various examples, at least one of Ra, Rb or Rc may be the remaining portion of the copolymer chain of the hydrogel polymer or polymeric nanogel particles. In alternative embodiments, one thiophene moiety may be in one set of copolymer chains and another thiophene in a second set of copolymer chains such that the photocycloadditions connects copolymer chains together or connects copolymer chains to functionalized surfaces of a substrate.

In various examples, photochemically-reversible hydrogel polymers and polymeric nanogel particles may comprise copolymer chains having hemithioindigo moieties capable of photoisomerization between E and Z isomers in accordance with the following scheme:

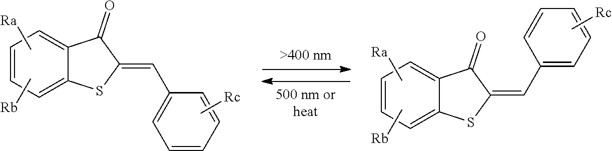

In various examples, the photoisomerization between E and Z isomers can be used to shift copolymer chains off surfaces, such as through electrostatic changes effecting bonding of monolayers. In various embodiments, Ra, Rb, Rc substituents on the hemithioindigo moieties may include oleyl amides or esters, wherein the oleyl chain provides van der Waals interactions that support self-assembled monolayers (SAMs). A substituent Ra, Rb, Rc may be the remaining portion of the copolymer chain of the hydrogel polymer or polymeric nanogel particle. Or two of Ra, Rb, Rc may be cyclized as part of the same or different polymer chain.

In various examples, photochemically-reversible hydrogel polymers and polymeric nanogel particles may comprise copolymer chains having doner-acceptor Stenhouse adducts capable of reversible cycloaddition in accordance with the following scheme:

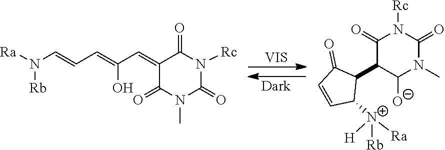

In various examples, the reversible photocycloaddition in the above scheme can be used to attach/detach hydrogel polymers or polymeric nanogel particles to/from surfaces, such as by altering steric or electrostatic interactions between polymer and surface. In various examples, at least one of Ra, Rb or Rc may be the remaining portion of the copolymer chain of the hydrogel polymer or polymeric nanogel particles.

In various examples, photochemically-reversible hydrogel polymers and polymeric nanogel particles may comprise copolymer chains having aryl substituted bis-imidazole capable of photochromism to imidazole radical species in accordance with the following scheme:

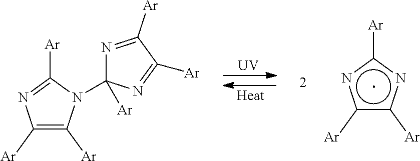

In various examples, the reversible reaction in the above scheme can be used to attach/detach hydrogel polymers or polymeric nanogel particles to/from surfaces, or to crosslink copolymer chains. In various examples, the aryl substituents may connect to the copolymer chains of the hydrogel polymer or polymeric nanogel particles.

To further illustrate the present disclosure, examples are provided below. These examples are provided for illustrative purposes and are Pot to be construed as limiting the scope of the disclosure in any way.

### EXAMPLES

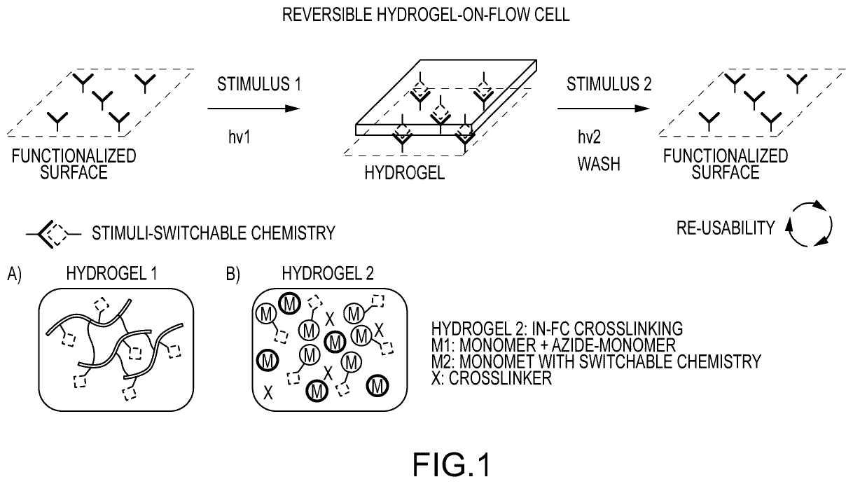

With reference now to FIG. 1, a photochemically-reversible hydrogel is reversibly attached to a suitably functionalized surface in a flow cell to be used for nucleic acid sequencing and then removed after the sequencing is complete. In this way, the photochemically-reversible hydrogel enables the FC to be reusable.

The top portion of FIG. 1 illustrates that with stimulus 1 (hυ1), a photochemically-reversible hydrogel in accordance with various examples of the present disclosure attaches to the suitably functionalized FC surface through a plurality of photoaddition reactions, e.g., [2+2] photodimerization. The FC surface comprises a plurality of tethered reactive alkene or 1,4-diene groups so that attachment of the hydrogel to the surface is achieved by photoaddition. Preferably, the radiation (hυ1) for attachment of the hydrogel to the functionalized surface is of wavelength >270 nm. The precise wavelength used for attachment of the hydrogel depends on whether alkenes or 1,4-dienes are used, and the electron density of the alkene or 1,4-diene functionalities, such as if certain electron-withdrawing or electron-donating groups are bonded to the alkene or 1,4-diene moieties. In certain examples, 4-methylcoumarin substituents both on hydrogel copolymer chains and tethered to the FC surface participate in photodimerization to attach the hydrogel to the FC surface.

The top portion of FIG. 1 also illustrates the removal of the hydrogel from the FC surface so that the FC can be reused. As illustrated, stimulus 2 (hυ2) causes cleavage of the dimers or [2+2+2+2] adducts so that the detached hydrogel can be easily washed away under mild conditions.

The lower portion of FIG. 1 shows two methods of achieving the reversible bonding of hydrogel to a FC surface. Method A) is as explained above—preformed hydrogel polymer is provided to the functionalized FC surface and the irradiated to dimerize alkene moieties or produce [2+2+2+2] cycloaddition adducts, bonding the hydrogel to the functionalized FC surface. In method B), a monomer mixture is provided to the FC surface, and polymerization is initiated on the surface. The is termed “in-FC crosslinking.” This example shows use of at least a monomer M1 having azide functionality (e.g., AzAPA), and at least a monomer M2 having reactive alkene or 1,4-diene functionality (e.g., CAA). A crosslinking compound is also added to the mixture prior to irradiation, such as BisAM. In this way, in situ polymerization forms the hydrogel polymer on the FC surface.

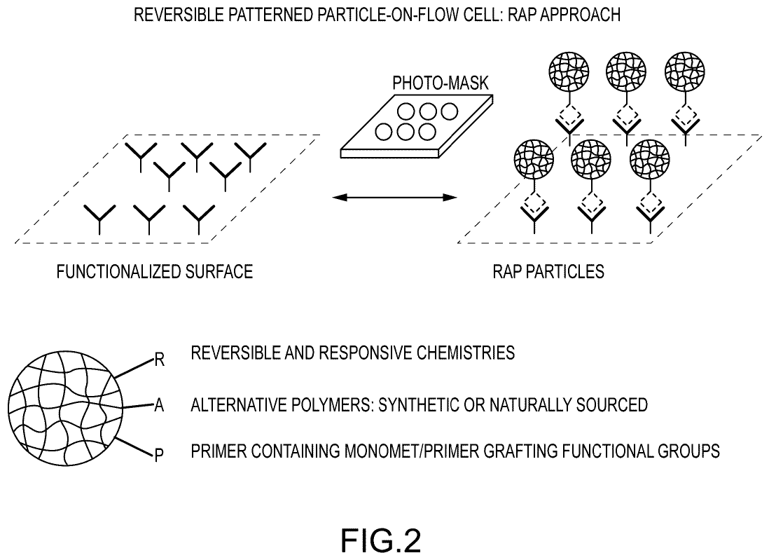

FIG. 2 provides an example of reversible patterned nanogel particles on a FC, termed “RAP approach.” “R” stands for reversible and responsive chemistries, such as provided by the presence of reactive alkene or reactive 1,4-diene end groups on copolymer chains of the nanogel particles. “A” stands for alternative polymers that are synthetic or naturally sourced. “P” stands for primer containing monomer having primer grating functional groups, such as —N₃ or —C≡CH groups that can participate in click-chemistry grafting of suitably functionalized amplification primers. In the example illustrated in FIG. 2, photochemically-reversible nanogel particles, such as RAP particles, are patterned onto a functionalized FC surface using a photomask.

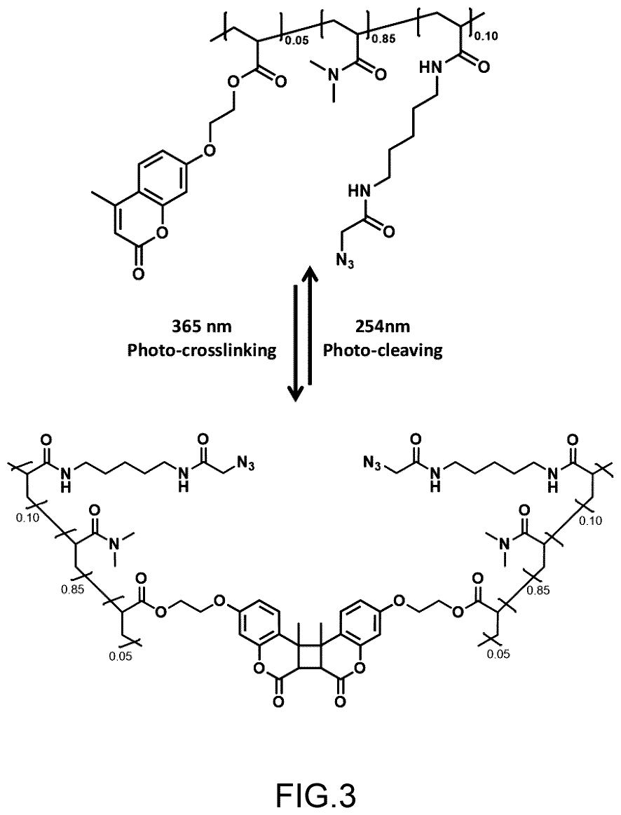

In the example illustrated in FIG. 3, photochemically-reversible hydrogels or nanogel particles having poly(CAA-co-NDMAM-co-AzAPA) copolymer chains can be reversibly crosslinked and cleaved by exposure to wavelengths of 365 nm and 254 nm, respectively. This example illustrates the photodimerization of the 4-methylcoumarin end groups in the copolymer chains of the hydrogel or nanogel particles.

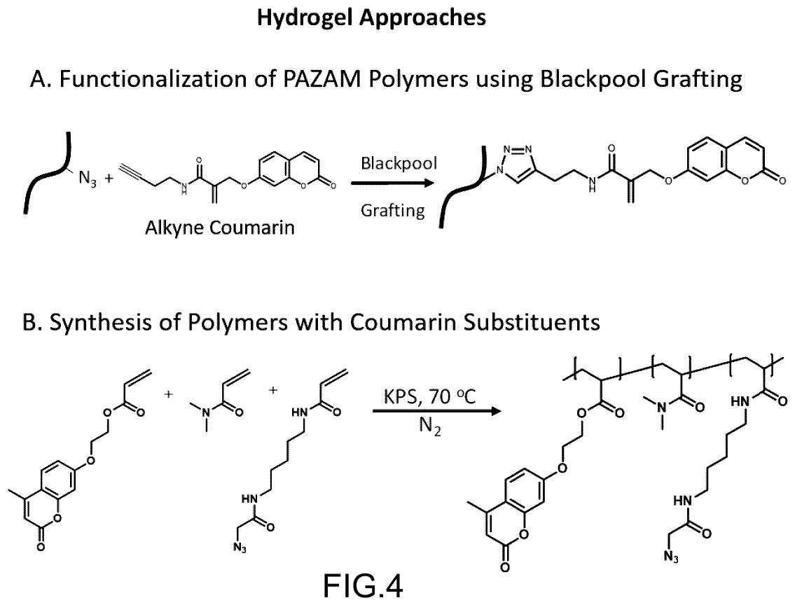

FIG. 4 illustrates synthetic routes to photochemically-reversible hydrogels or nanogel particles. In method A, PAZAM or other polymers comprising copolymer chains having —N₃ end groups are reacted with alkyne coumarin under copper catalysis to form photochemically-reversible hydrogels or nanogel particles having tethered coumarin substituents. In method A, the photochemically-reversible hydrogels or nanogel particles do not begin as such but end up having reactive alkene end groups on copolymer chains after the reaction to link the coumarin tethers to the hydrogel or nanogel particles through triazine linkers.

In the exemplary method B illustrated in FIG. 4, photochemically-reversible hydrogels or nanogel particles are synthesized directly by free radical polymerization of a monomer mixture consisting of CAA, NDMAM and AzAPA. The free radical initiator here is potassium persulfate (KPS), and the reaction is conducted under an inert atmosphere at 70° C. The resulting photochemically-reversible hydrogels or nanogel particles comprise poly(CAA-co-NDMAM-co -AzAPA) copolymer chains, and thus have dual functionality by the presence of both reactive alkene end groups and —N₃ end groups on at least some of the copolymer chains. Without the presence of —CO₂H end groups (AAc was not used for this copolymer), attachment of the photochemically-reversible hydrogel or nanogel particles to a FC is by way of photodimerization.

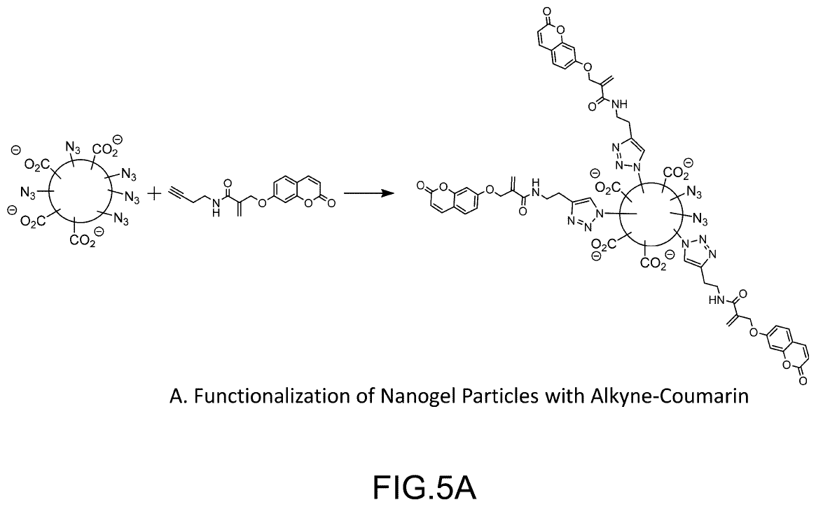

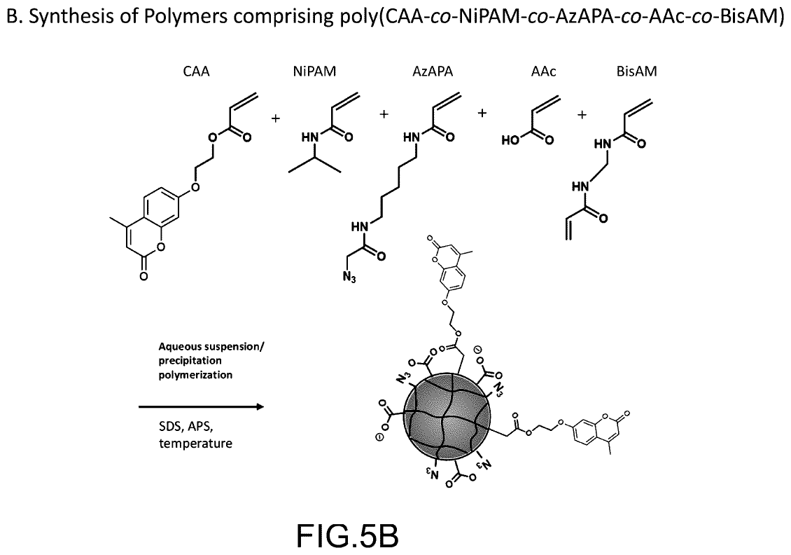

In the example illustrated in FIG. 5A, photochemically-reversible nanogel particles are prepared by reacting nanogel particles comprising —N₃ end groups on at least some of the copolymer chains with alkyne coumarin. In this way, reactive coumarin groups are tethered onto previously formed nanogel particles to make photochemically-reversible nanogel particles. Although exemplified with nanogel particles, the same process can be used to tether coumarin groups onto hydrogel polymers comprising —N₃ end groups on at least some of its copolymer chains.

In the example illustrated in FIG. 5B, photochemically-reversible nanogel particles are directly prepared from a monomer mixture of CAA, NiPAM, AzAPA, AAc and BisAM under aqueous suspension/precipitation free-radical polymerization conditions. In this example, ammonium persulfate (APS) is used as the free-radical initiator, and sodium dodecyl sulfate (SDS) as the dispersant. The resulting photochemically-reversible nanogel particle illustrated comprises poly(CAA-co-NiPAM-co-AzAPA-co-AAc-co-BisAM) copolymer chains, wherein at least some of the copolymer chains have coumarin, —N₃, and —CO₂H end groups.

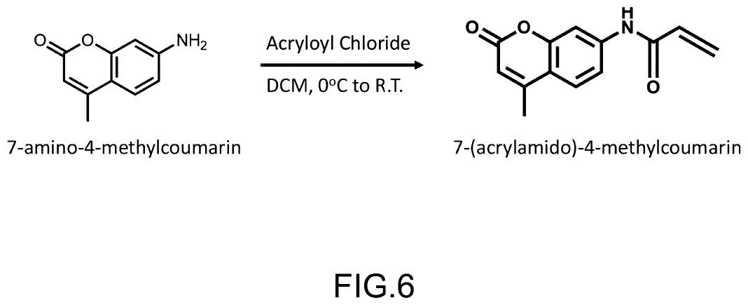

In the example illustrated in FIG. 6, N-(4-methyl-2-oxo-2H-chromen-7-yl)acrylamide (7-(acrylamido0-4-methylcoumarin) is prepared by reacting 7-amino-4-methylcoumarin with acryloyl chloride in dichloromethane (DCM) at from 0° C. to ambient. Although not exemplified, 4-methyl-2-oxo-2H-chromen-7-yl acrylate can be similarly prepared beginning with 7-hydroxy-4-methylcoumarin.

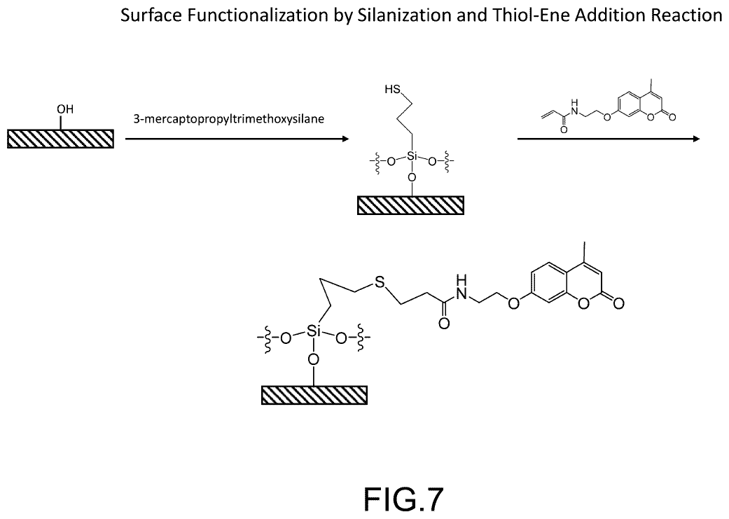

In the example illustrated in FIG. 7, a FC surface is functionalized by sequential treatment with 3-mercaptoproplytirmethoxysilane and N-(2-((4-methyl-2-oxo-2H-chromen-7-yl)oxy)ethyl)acrylamide. The resulting FC surface is characterized as having tethered coumarin groups (or more accurately, tethered 4-methylcoumarin groups). Photochemically-reversible hydrogels or nanogel particles are then provided in solution to this functionalized surface, followed by irradiation of the surface to bind the hydrogel or plurality of nanogel particles to the functionalized surface by photodimerization of coumarin groups.

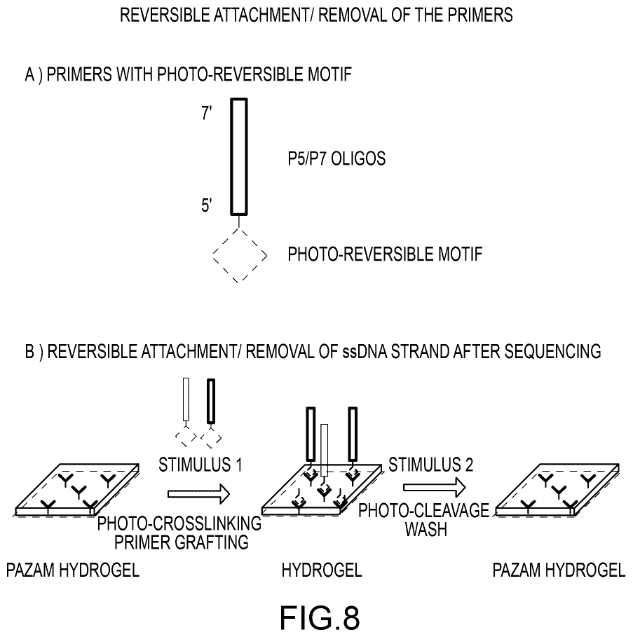

In the example illustrated in FIG. 8, P5/P7 amplification primers are functionalized at the 5′-end with photo-reversible motifs, such as with reactive alkene or reactive 1,4-diene containing substituents. This example replaces grafting of functionalized P5/P7 amplification primers to hydrogels or nanogel particles through alkyne—N₃ or N₃-alkyne click-chemistry and is reversible through photochemistry. As illustrated in b), a PAZAM hydrogel on a FC surface may also comprise photo-reversible motifs, such that the functionalized P5/P7 amplification primers are reversibly attached to the hydrogel layer by photodimerization. Once the nucleic acid sequencing experiment is complete, irradiation at <300 nm cleaves off the primers and a simple wash restores the hydrogel coated surface.

In the detailed description, references to “various examples”, “one example”, “an example”, etc., indicate that the example described may include a particular feature, structure, or characteristic, but every example may not necessarily include the particular feature, structure, or characteristic. Moreover, such phrases are not necessarily referring to the same example. Further, when a particular feature, structure, or characteristic is described in connection with an example, it is submitted that it is within the knowledge of one skilled in the art to affect such feature, structure, or characteristic in connection with other examples whether or not explicitly described. After reading the description, it will be apparent to one skilled in the relevant art(s) how to implement the disclosure in alternative examples.

Benefits, other advantages, and solutions to problems have been described herein with regard to specific examples. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an example, B alone may be present in an example, C alone may be present in an example, or that any combination of the elements A, B and C may be present in a single example; for example, A and B, A and C, B and C, or A and B and C.

All structural, chemical, and functional equivalents to the elements of the above-described various examples that are known to those of ordinary skill in the art are expressly incorporated herein by reference and are intended to be encompassed by the present claims. Moreover, it is not necessary for an apparatus or component of an apparatus, or method in using an apparatus to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.

1. A hydrogel polymer comprising copolymer chains further comprising:
a first recurring unit of Formula (I): 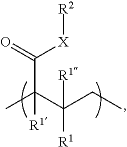
wherein:
each of R¹, R^{1′}, and R^{1″} is independently selected from H, halogen, alkyl, alkoxy, alkenyl, alkynyl, cycloalkyl, aryl, heteroaryl, or heterocyclyl;
X is —O— or —NH—; and
R² is —CH₂—C≡CH, or R² has a structure: 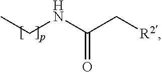
wherein R^{2′} is —NH₂, alkyl, alkoxy, alkenyl, alkynyl, or optionally substituted variants thereof, or halogen, —N₃, —OH, —C(O)H, —NH═NH₂, —SCN, —CO₂H, —SH, glycidyl, epoxy, aziridine, triazoline, or —C≡CH; and
p is an integer of 1 to 50; and
a second recurring unit of Formula (II), 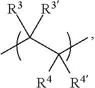
wherein:
each of R³, R^{3′}, R⁴, and R^{4′} is independently selected from —H, —R⁵, —OR⁵, —CO₂R⁵, —C(O)R⁵, —OC(O)R⁵, —C(O)NR⁶R⁷, —NR⁶R⁷ or a substructure of Formula (III), 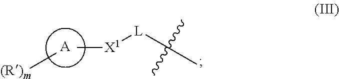
R⁵ is —H, —OH, alkyl, cycloalkyl, hydroxyalkyl, aryl, heteroaryl, or heterocyclyl;
each of R⁶ and R⁷ is independently selected from —H and alkyl;
A is an aryl or a thymidinyl moiety;
R′ is —H, alkyl, alkoxy, alkenyl, alkynyl, aryl, heterocyclyl or optionally substituted variants thereof, or halogen, —N₃, —OH, —C(O)H, —NH═NH₂, —SCN, —CO₂H, —SH, glycidyl, epoxy, or two R′ groups, when bonded to adjacent atoms on ring A and taken together with ring A, form a coumarinyl, an anthracenyl, an acenaphthylenyl, a thianaphthenyl-1-oxide or a thianaphthenyl-1,1-dioxide moiety;
X¹ is a bond, —(CH₂)_{q}—, —O— or —NH—;
L is a bivalent linker having a structure —(CH₂)_{q}—X²—C(═O)— or —(CH₂CH₂O)_{q}—X²—C(═O)—;
X² is —O— or —NH—;
m is an integer from 1 to 9; and
q is an integer from 0 to 50;
wherein at least some of the copolymer chains include at least one reactive alkene or reactive 1,4-diene end group capable of [2+2] or [2+2+2+2] photodimerization, respectively, at wavelengths >270 nm.

2. The hydrogel polymer of claim 1, wherein at least some of the copolymer chains include at least one N₃, —C≡CH or —CO₂H end group.

3. The hydrogel polymer of claim 1, wherein Formula (III) comprises the subgenus structure of Formula (IV): 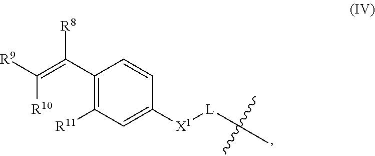
wherein each of R⁸ and R⁹ is —H, alkyl, cycloalkyl, hydroxyalkyl, aryl, heteroaryl, or heterocyclyl; each R¹⁰ and R¹¹ is independently —H, alkyl, cycloalkyl, hydroxyalkyl, aryl, heteroaryl, or heterocyclyl, or R¹⁰ is —C(═O)—, is —O—, and R¹⁰ and R¹¹ are bonded together such that Formula (IV) comprises a substituted coumarin moiety.

4. The hydrogel polymer of claim 1, wherein the recurring unit of Formula (I) is:

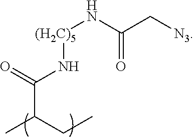

5. The hydrogel polymer of claim 1, wherein the recurring unit of Formula (II) is:

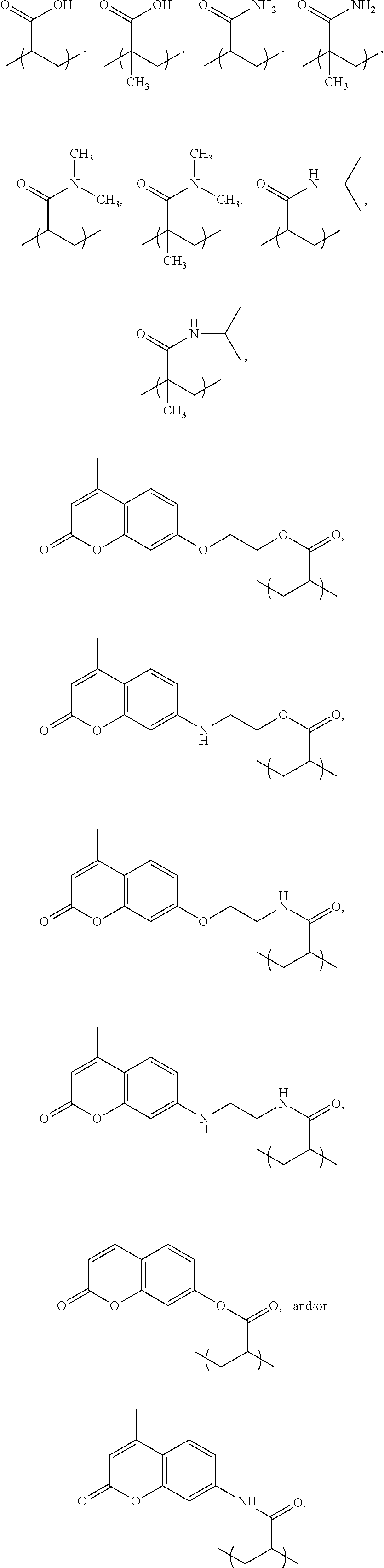

6. The hydrogel polymer of claim 1, wherein the recurring unit of Formula (II) is: 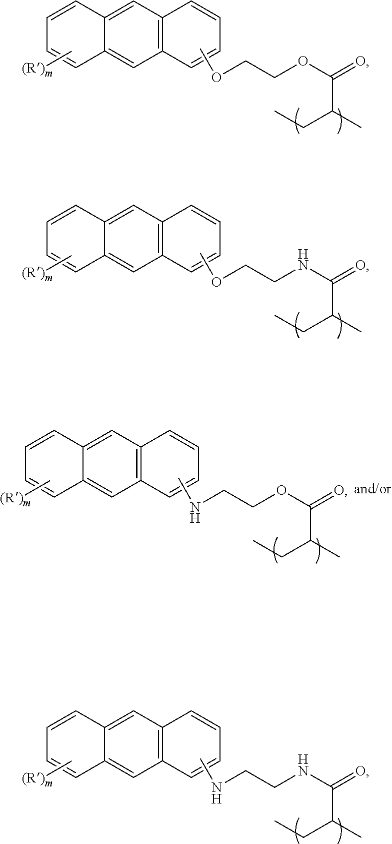
wherein R′ is —H, alkyl, alkoxy, alkenyl, alkynyl, aryl, heterocyclyl or optionally substituted variants thereof, or halogen, —N₃, —OH, —C(O)H, —NH═NH₂, —SCN, —CO₂H, —SH, glycidyl, epoxy; and,
m is an integer from 1 to 9.

7. The hydrogel polymer of claim 1, wherein the recurring unit of Formula (II) is: 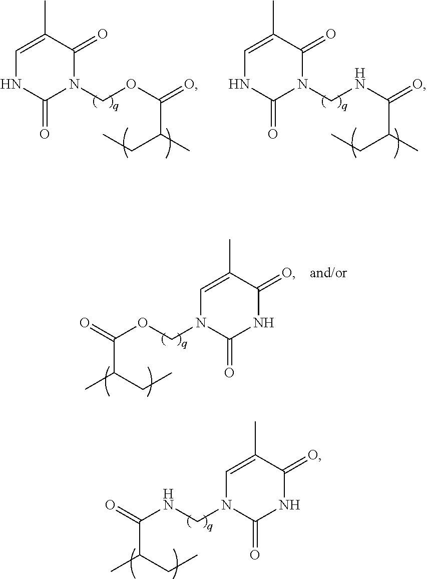
wherein q is an integer from 0 to 50.

8. The hydrogel polymer of claim 1, wherein the recurring unit of Formula (II) is: 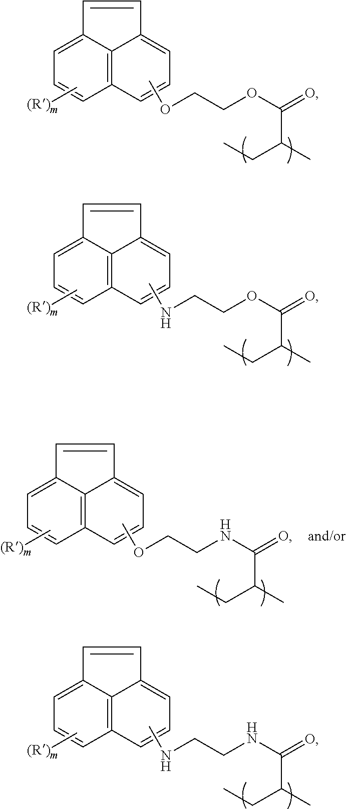
wherein R′ is —H, alkyl, alkoxy, alkenyl, alkynyl, aryl, heterocyclyl or optionally substituted variants thereof, or halogen, —N₃, —OH, —C(O)H, —NH═NH₂, —SCN, —CO₂H, —SH, glycidyl, epoxy; and,
m is an integer from 1 to 7.

9. The hydrogel polymer of claim 1, wherein the recurring unit of Formula (II) is: 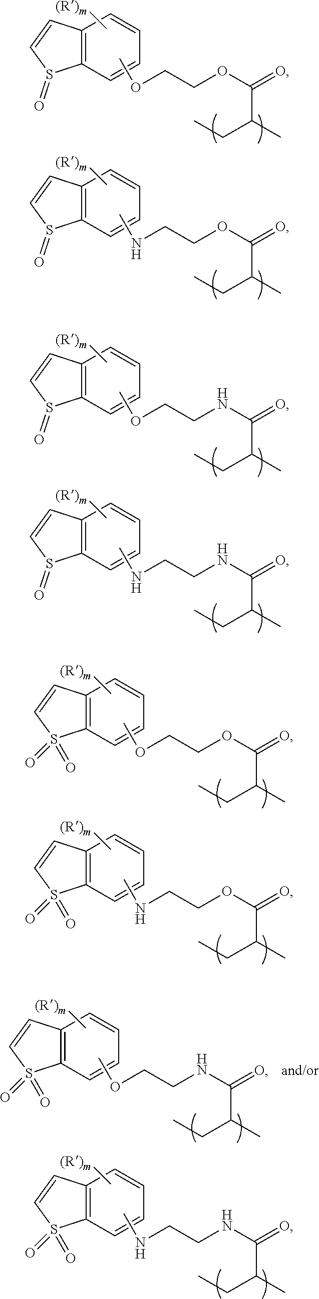
wherein R′ is —H, alkyl, alkoxy, alkenyl, alkynyl, aryl, heterocyclyl or optionally substituted variants thereof, or halogen, —N₃, —OH, —C(O)H, —NH═NH₂, —SCN, —CO₂H, —SH, glycidyl, epoxy; and,
m is an integer from 1 to 5.

10. The hydrogel polymer of claim 1, wherein the recurring unit of Formula (II) is:

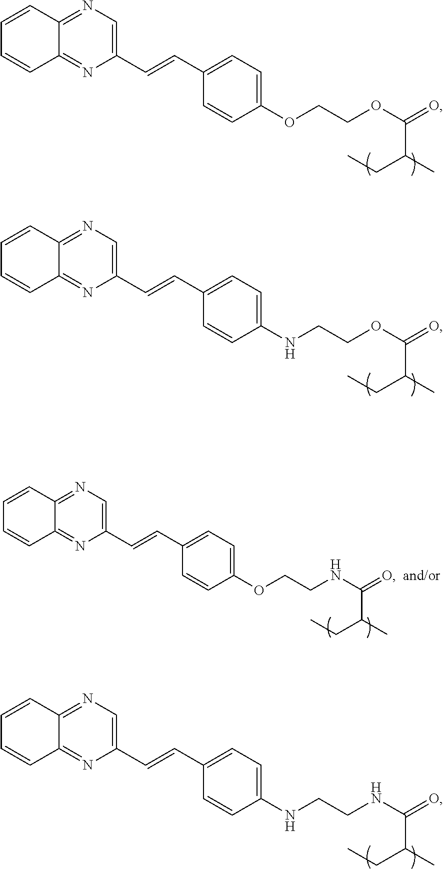

11. The hydrogel polymer of claim 3, wherein either R⁹ or R¹⁰ is —CO₂H, such that Formula (IV) is a cis- or trans-cinnamyl acid moiety.

12. The hydrogel polymer of claim 3, wherein R⁹ or R¹⁰ is aryl, such that Formula (IV) is a cis- or trans-stilbene moiety.

13. The hydrogel polymer of claim 1, derived from a monomer mixture comprising: 7-((2-methacryloyloxy)ethoxy)-4-methylcoumarin; N-isopropylacrylamide; N-(5-(2-azidoacetamido)pentyl)acrylamide; and acrylic acid.

14. The hydrogel polymer of claim 1, derived from a monomer mixture comprising: 7-((2-methacryloyloxy)ethoxy)-4-methylcoumarin; N,N-dimethylacrylamide; N-(5-(2-azidoacetamido)pentyl)acrylamide; and acrylic acid.

15. The hydrogel polymer of claim 1, derived from a monomer mixture comprising: 7-((2-acrylamido)ethoxy)-4-methylcoumarin; N-isopropylacrylamide; N-(5-(2-azidoacetamido)pentyl)acrylamide; and acrylic acid.

16. The hydrogel polymer of claim 1, derived from a monomer mixture comprising: 7-((2-acrylamido)ethoxy)-4-methylcoumarin; N,N-dimethylacrylamide; N-(5-(2-azidoacetamido)pentyl)acrylamide; and acrylic acid.

17. The hydrogel polymer of claim 1, derived from a monomer mixture comprising: 7-(acrylamido)-4-methylcoumarin; N-isopropylacrylamide; N-(5-(2-azidoacetamido)pentyl)acrylamide; and acrylic acid.

18. The hydrogel polymer of claim 1, derived from a monomer mixture comprising: 7-(acrylamido)-4-methylcoumarin; N,N-dimethylacrylamide; N-(5-(2-azidoacetamido)pentyl)acrylamide; and acrylic acid.

19. The hydrogel polymer of claim 1, derived from a monomer mixture comprising: 7-(methacrylamido)-4-methylcoumarin; N-isopropylacrylamide; N-(5-(2-azidoacetamido)pentyl)acrylamide; and acrylic acid.

20. The hydrogel polymer of claim 1, derived from a monomer mixture comprising: 7-(methacrylamido)-4-methylcoumarin; N,N-dimethylacrylamide; N-(5-(2-azidoacetamido)pentyl)acrylamide; and acrylic acid.

21. The hydrogel polymer of claim 1, derived from a monomer mixture comprising: 7-(acryloyloxy)-4-methylcoumarin; N-isopropylacrylamide; N-(5-(2-azidoacetamido)pentyl)acrylamide; and acrylic acid.

22. The hydrogel polymer of claim 1, derived from a monomer mixture comprising: 7-(acryloyloxy)-4-methylcoumarin; N,N-dimethylacrylamide; N-(5-(2-azidoacetamido)pentyl)acrylamide; and acrylic acid.

23. The hydrogel polymer of claim 1, derived from a monomer mixture comprising: 7-(methacryloyloxy)-4-methylcoumarin; N-isopropylacrylamide; N-(5-(2-azidoacetamido)pentyl)acrylamide; and acrylic acid.

24. The hydrogel polymer of claim 1, derived from a monomer mixture comprising: 7-(methacryloyloxy)-4-methylcoumarin; N,N-dimethylacrylamide; N-(5-(2-azidoacetamido)pentyl)acrylamide; and acrylic acid.

25. The hydrogel polymer of claim 13, wherein the monomer mixture further comprises a multi-functional compound selected from the group consisting of N,N′-methylenebisacrylamide, N,N′-methylenebismethacrylamide, polyethylene glycol diacrylate, polyethylene glycol dimethacrylate, N-vinylacrylamide, glycidyl acrylate, divinyibenzene, tetraallyl ammonium chloride, and mixtures thereof.

26. The hydrogel polymer of claim 1, wherein the hydrogel polymer is in the form of nanogel particles.

27. The hydrogel polymer of claim 1, wherein the hydrogel polymer further comprises amplification primers conjugated thereon.

28. The hydrogel polymer of claim 27, wherein each conjugation between an amplification primer and the hydrogel polymer comprises click-chemistry between a terminal alkyne substituent on the amplification primer and an azide group on an end of a respective copolymer chain or click-chemistry between a terminal azide substituent on the amplification primer and an alkyne group on an end of a respective copolymer chain.

29. The hydrogel polymer of claim 1, wherein at least some of the copolymer chains are crosslinked by photo-dimerization between the reactive alkene or reactive 1,4-diene end groups capable of [2+2] or [2+2+2+2] photodimerization, respectively, at wavelengths >270 nm.

30.-50. (canceled)
